SEQUENCE LISTING (2 sequences)

SEQ ID NO: 1 - DNA, 11 bp, synthetic construct
  source 1..11: mol_type = other DNA
gaagagcgtcg

SEQ ID NO: 2 - DNA, 22 bp, synthetic construct
  source 1..22: mol_type = other DNA
tagggaaagagtgtagatctcg

(19) United States
(12) Patent Application Publication
(10) Pub. No.: US 2024/0409918 A1
(43) Pub. Date: Dec. 12, 2024

(54) SYSTEMS AND METHODS FOR ISOLATION OF DESIRED NUCLEIC ACID STRANDS

(71) Applicant: The Trustees of Columbia University in the City of New York, New York, NY (US)

(72) Inventors: Peter Alan Sims, Ardsley, NY (US); Hovig Bayandorian, Berkeley, CA (US)

(21) Appl. No.: 18/712,168

(22) PCT Filed: Nov. 22, 2022
(86) PCT No.: PCT/US22/80304

§ 371 Date: May 21, 2024

Related U.S. Application Data
(60) Provisional application No. 63/281,807, filed on Nov. 22, 2021.

(51) Int. Cl. C12N 15/10; C12Q 1/6806; C12Q 1/6869; G16B 30/00

(52) U.S. Cl. CPC C12N 15/1006; C12N 15/1089; C12Q 1/6806; C12Q 1/6869; G16B 30/00

(57) ABSTRACT

The present disclosure provides systems and methods for isolation of nucleic acids. In particular, provided herein are nanopore-based or substrate-based sequencing systems and methods of use thereof for isolation of desired nucleic acids from a mixed library containing both accurate and inaccurate strands.

### STATEMENT REGARDING RELATED APPLICATIONS

This application claims priority to U.S. Provisional Patent Application No. 63/281,807, filed Nov. 22, 2021, the entire contents of which are incorporated herein by reference for all purposes.

### SEQUENCE LISTING

The text of the computer readable sequence listing filed herewith, titled “COLUM-39834-601_SQL”, created Nov. 22, 2022, having a file size of 2,875 bytes, is hereby incorporated by reference in its entirety.

### FIELD

The present disclosure provides systems and methods for isolation of desired nucleic acid strands from a sample containing nucleic acid strands.

### BACKGROUND

In applications involving work with nucleic acid strands, it can be useful to be able to physically separate nucleic strands which meet a specific desired property or combination(s) of desired properties. For example, in nucleic acid sequencing applications, it may be desirable to isolate nucleic acid strands of a specific length range. As another example, during the process of nucleic acid synthesis, synthesized strands may be inaccurate or of the wrong length, in which case isolation of accurate strands of the correct length and/or sequence identity may be desirable. As another example, in targeted sequencing applications, it may be desirable to isolate nucleic acid strands which have a particular sequence identity. Described herein are systems and methods for isolating nucleic acid strands which meet a specified desired property or combination(s) of desired properties, including but not limited to sequence identity, approximate sequence identity, length, approximate length, methylation status, and hybridization to proteins or nucleic acid probes.

Processes for nucleic acid synthesis, such as phosphoramidite chemistry nucleic acid synthesis, may produce a significant portion of nucleic acid strands having errors. For example, processes may produce a significant portion of nucleic acid strands of an incorrect length, containing one or more insertion/deletion errors, and/or containing one or more single point mutation errors. Accordingly, it is desirable to have a process which can inspect the product of the nucleic acid synthesis reaction and isolate the intended (e.g. accurate) synthesis product. Currently, the preferred technique for performing this isolation is molecular cloning followed by sequencing of clonally amplified nucleic acid synthesis product. However, this process requires between one and three weeks and comes at considerable cost. Accordingly, there is a need for rapid, cost-efficient methods for synthesis and subsequent isolation of accurate nucleic acid products.

### SUMMARY

In some aspects, provided herein are methods of separating desired nucleic acid molecules from a sample containing nucleic acids. A “desired” nucleic acid molecule refers to a nucleic acid strand of which isolation is intended. The “desired” nucleic acid molecule may be an “accurate” nucleic acid strand, or it may be an “inaccurate” nucleic acid strand. An “accurate′ nucleic acid strand refers to a strand determined to have an intended property, such as having a specific sequence identity, length, methylation status, other modification, or other property which may be selected by a user, whereas an “inaccurate” nucleic acid refers to strand determined to not have the intended property.

In some embodiments, provided herein are methods of separating desired nucleic acid molecules from undesired nucleic acid molecules contained in a mixed library of nucleic acid molecules. In some embodiments, the method comprises sequencing individual nucleic acid molecules within said mixed library at a localized zone of a device. In some embodiments, the method further comprises selectively separating desired nucleic acid from undesired nucleic acid by releasing either the desired or the undesired nucleic acid from said localized zone based on its determined sequence. For example, in some embodiments the method comprises releasing the desired nucleic acid molecules from the localized zone of the device. As another example, in some embodiments the method comprises releasing the undesired nucleic acid molecules from the localized zone of the device. In some embodiments, the nucleic acid molecules are synthesized nucleic acid molecules. In some embodiments, the method comprises separating a first population of desired nucleic acid molecules into a first sub-library, and separating a second population of desired nucleic acid molecules into a second sub-library. For example, a first population of accurate nucleic acid strands may be released from the localized zone of the device and collected to generate a first sub-library of desired nucleic acid strands. Subsequently, a second population of accurate nucleic acid strands may be released from the localized zone of the device and collected to generate a second sub-library of desired nucleic acid strands.

In some aspects, provided herein are methods of isolating nucleic acid strands from a mixed library. In some embodiments, the method comprises providing a sample containing the mixed library to a first chamber of a nanopore sequencing device. In some embodiments, the device comprises a first chamber and a second chamber separated by a substantially impermeable membrane. In some embodiments, the substantially impermeable membrane houses a plurality of nanopores. In some embodiments, the method comprises inducing a flow of current through each nanopore, such that individual nucleic acid strands enter into the nanopores housed within the membrane. In some embodiments, the method comprises determining whether a given nucleic acid strand passing through a nanopore is accurate or inaccurate. For example, the method may comprise determining whether a given nucleic acid strand passing through a nanopore has an accurate sequence, an accurate length, an accurate methylation status, and/or another property. In some embodiments, the method comprises determining the sequence of each individual nucleic acid strand as it passes through a nanopore and identifying each strand as accurate or inaccurate. In some embodiments, the method further comprises isolating the desired nucleic acid strands from the sample. The desired nucleic acid strands may be accurate nucleic acid strands or inaccurate nucleic acid strands, depending on the intended method to be employed.

In some embodiments, the nanopore sequencing device comprises a plurality of electrodes. In some embodiments, each electrode is operably connected to a distinct nanopore within the substantially impermeable membrane. In some embodiments, inducing a flow of current through each nanopore comprises applying a voltage through each of the plurality of electrodes.

In some embodiments, the nanopore sequencing device further comprises a plurality of sensors. In some embodiments, each sensor records a current passing through a single nanopore, such that the current passing through each nanopore is independently recorded.

In some embodiments, determining whether a given strand passing through a nanopore is accurate or inaccurate involves recording the current passing through each nanopore. In some embodiments, determining whether a given nucleic acid is accurate or inaccurate involves recording the current passing through each nanopore and measuring the disruption of current that occurs as the nucleic acid strand passes through the nanopore. Disruption of the current can be used to determine whether the nucleic acid strand has one or more desired properties.

In some embodiments, determining the sequence of each individual nucleic acid strand as it passes through a nanopore involves recording the current passing through each nanopore. In some embodiments, determining the sequence of each individual nucleic acid as it passes through a nanopore involves recording the current passing through each nanopore and determining the sequence of a given nucleic acid strand based upon the disruption of current that occurs as the nucleic acid strand passes through the nanopore. In some embodiments, identifying each strand as accurate or inaccurate comprises comparing the determined sequence of a given nucleic acid strand to an accurate nucleic acid sequence. In some embodiments, a strand comprising one or more mutations compared to the accurate nucleic acid sequence is identified as an inaccurate strand.

In some embodiments, isolating the desired nucleic acid strands from the sample comprises modulating the voltage applied through each electrode operably connected to a nanopore containing an undesired nucleic acid strand, such that passage of the undesired nucleic acid strand through the nanopore is halted and/or reversed. In some embodiments, the voltage applied through each electrode operably connected to nanopore containing a desired nucleic acid strand is not modulated, such that desired nucleic acid strands pass through the nanopores into the second chamber of the nanopore sequencing device. In some embodiments, the method comprises isolating the desired nucleic acid strands from the second chamber of the nanopore sequencing device.

In some embodiments, the method further comprises reversing the voltage applied to each electrode operably connected to a nanopore containing an undesired nucleic acid strand, such that the undesired nucleic acid strands are ejected into the first chamber of the nanopore sequencing device. In some embodiments, the method further comprises removing the undesired nucleic acid strands from the first chamber. In some embodiments, following removal of the undesired nucleic acid strands from the first chamber the voltage applied to one or more electrodes is reversed, such that the desired nucleic acid strands housed within the second chamber are drawn through the nanopores into the first chamber. In some embodiments, the method further comprises removing the desired nucleic acid strands from the first chamber.

In some embodiments, one or more steps of the methods described herein are performed using a computer.

In some embodiments, provided herein are methods of isolating desired nucleic acid strands from a mixed library based upon substrate-based sequencing. In some embodiments, methods of isolating desired nucleic acid strands from a mixed library comprise providing a sample comprising the mixed library to a substrate. In some embodiments, the substrate comprises a plurality of cleavable anchors at distinct locations on the surface of the substrate. In some embodiments, individual nucleic acid strands bind to the cleavable linkers. In some embodiments, the method comprises applying a stimulus to induce selective cleavage of the cleavable anchors bound to desired locations on the substrate, thereby releasing desired nucleic acid strands, if present, from those spatial locations on the substrate.

In some embodiments, the method further comprises identifying each strand as accurate or inaccurate. For example, the method may comprise identifying whether each strand possesses a desired sequence, length, methylation status, or other property. In some embodiments, the method comprises determining the sequence the nucleic acid strands and identifying each strand as accurate or inaccurate.

In some embodiments, the method further comprises applying a stimulus to induce selective cleavage of the cleavable anchors bound to desired nucleic acid strands, thereby releasing desired nucleic acid strands from the surface of the substrate. In some embodiments, the method further comprises isolating the released nucleic acid strands.

In some embodiments, identifying each strand as accurate or inaccurate comprises comparing the determined sequence of a given nucleic acid strand to an accurate nucleic acid sequence. In some embodiments, a strand comprising one or more mutations compared to the accurate nucleic acid sequence is identified as an inaccurate strand.

In some embodiments, the cleavable anchors are photocleavable. In such embodiments, the stimulus to induce selective cleavage may be light. For example, the light may be ultraviolet light. In some embodiments, the cleavable anchors are heat cleavable. In such embodiments, the stimulus to induce selective cleavage may comprise heat. The stimulus may be delivered in a spatially selective manner to the substrate. For example, the stimulus may be applied to a specific spatial location on the substrate.

In some aspects, provided herein are systems for isolating desired nucleic acid strands from a sample containing nucleic acids. In some embodiments, provided herein is a system for isolating desired nucleic acid strands from a mixed library. In some embodiments, the system comprises a sequencing device and software. In some embodiments, the software collects data from the sequencing device, analyzes the data, and actuates components of the system to control the isolation of accurate nucleic acids from the mixed library. In some embodiments, collecting data comprises determining whether a given nucleic acid present at a localized zone of the sequencing device is accurate or inaccurate. For example, collecting data may comprise determining whether a nucleic acid strand has a desired sequence, length, methylation status, or other property. In some embodiments, analyzing the data comprises comparing the property of the nucleic acid (e.g. length, sequence methylation status, etc.) to that of a known, desired nucleic acid strand. In some embodiments, collecting data comprises determining the sequence of a nucleic acid at a localized zone of the sequencing device. In some embodiments, analyzing the data comprises comparing the sequence of the nucleic acid to the sequence of a desired nucleic acid strand.

In some embodiments, the software encodes machine readable instructions that instruct a processor to execute a given task to control the isolation of accurate nucleic acids from the mixed library. In some embodiments, the software encodes machine readable instructions that instruct a processor to apply a stimulus that results in selective release of either a desired or an undesired nucleic acid strand from the localized zone of the sequencing device.

In some embodiments, the sequencing device is a nanopore based sequencing device. In some embodiments, the software encodes machine readable instructions that instruct a processor to apply a voltage or to modulate voltage at a given electrode of the nanopore based sequencing device, thereby selectively releasing either a desired or an undesired nucleic acid strand from a nanopore operably connected to the electrode.

In some embodiments, the sequencing device is a substrate-based sequencing device. In some embodiments, the software encodes machine readable instructions that instruct a processor to apply an ultraviolet light to a defined spatial location on a substrate, thereby releasing either a desired or an undesired nucleic acid strand from the defined spatial location on the substrate.

Other aspects and embodiments of the disclosure will be apparent in light of the following detailed description.

### BRIEF DESCRIPTION OF THE DRAWINGS

FIG. 1 shows a schematic of an exemplary method for accurate DNA strand isolation through controlled translation during nanopore sequencing. In the depicted method, a nanopore sequencing device is used to generate raw signal for the sequencing of synthesized nucleic acid strands as they pass through a nanopore. The same nucleic strand may be sent backwards and forwards through the nanopore to generate redundant reads of the same molecule. A computerized process combines these one or more reads of the strand along with prior information about the desired sequence and possible barcoding or error-correcting schemes to generate a decision as to whether the strand is desired. If the strand is desired it may be either permitted to pass through the nanopore or it may be held in place by the nanopore. Accordingly, two physically separate fluid volumes are created, one volume containing desired strands and the other containing undesired strands. One potential method to perform this separation is to perform pipette aspiration of the cis chamber above the nanopore to remove potential contaminants and/or undesired strands that were not permitted passage through the chamber, followed by replenishment of some of the fluid volume in the cis chamber to facilitate flow of current, followed by reversal of the nanopore voltage to eject the desired strands into the cis chamber, followed by isolation (e.g. aspiration) of these desired strands.

FIG. 2 shows a schematic of another exemplary method for isolation of accurate DNA strands through arrayed substrate-based sequencing (SBS)-based DNA template identification and targeted isolation by UV photocleavage. The schematic shows a substrate-based sequencing (also referred to herein as “SBS”) technology, which may include any sequencing-by-synthesis or sequencing-by-binding technology. In a SBS, the location of the nucleic strand(s) being interrogated has/have a spatially localized position on a substrate. In this exemplary embodiment, an SBS method is used to determine the sequence and location of synthesized strands which are immobilized to a substrate with a photopolymer. A targeted illumination machine selectively exposes the substrate in locations corresponding resulting in the cleaving of the photopolymer, followed by a wash, to separate the accurate from inaccurate synthesized strands. A high-accuracy PCR step may be used following the photocleaving process to produce larger volumes of nucleic acid strand product.

FIG. 3 shows a schematic of another exemplary method for identification of accurate DNA template strands by single molecule real-time sequencing followed by targeted isolation with UV photocleavage. In some embodiments, a highly processive polymerase (e.g. DNA polymerase) is immobilized on a surface and bound to a template (e.g. DNA template). The polymerase incorporates nucleotides modified with fluorescent labels, and this process is monitored in real-time with a fluorescence detection system to sequence the template. The polymerase can be immobilized to surfaces using, for example, biotin-streptavidin bioconjugation chemistry. In some embodiments, biotinylation reagents that include photocleavable chemical linkers are used that allow release of biotinylated proteins from surfaces upon exposure to ultraviolet (UV) light. Accordingly, shown herein is an optical system that allows targeted release of individual polymerase-bound templates having the desired sequence by high-resolution direction of UV light.

FIG. 4 shows a schematic of another exemplary method for identification of accurate DNA template strands by SBS sequencing of clonally amplified DNA templates followed by targeted isolation with UV photocleavage. An SBS technology for sequencing small colonies of clonally amplified templates, rather than single molecules, could be employed for identification and selective release of accurate nucleic acid strands. In some embodiments of clonal SBS, individual template strands are captured on surface-immobilized oligonucleotide primers by hybridization and clonally amplified using surface-immobilized primers by solid-phase PCR (e.g. bridge PCR). A variety of sequencing chemistries can then be used to sequence the clonally amplified DNA templates, including, for example, the reversible terminator chemistry commercialized by Illumina. Oligonucleotide primers can be immobilized on surfaces (e.g. on the surface of the substrate) using photocleavable chemical linkers that allow targeted release of oligonucleotides and covalently conjugated templates by exposure to UV light. Thus, similar to the system described in FIG. 3, one an optical system that allows targeted release of individual template clonal amplicons by high-resolution direction of UV light can be used. The released material would include both oligonucleotide primers and the desired primer-conjugated DNA templates, which can be readily separated by size selection.

FIG. 5 shows three exemplary schematics for exposing a substrate-based sequencing substrate to light for the purposes of photocleaving a photosensitive linker. FIG. 5A depicts, from left to right, a light source (e.g. ultraviolet, infrared, or visible wavelength light) emitting light (depicted as purple arrows) into a lens assembly (in blue), which then focuses the light onto a digital micromirror array (or spatial light modulator), which then reflects (or transmits) the light into another lens assembly, which then focuses the light (purple arrows) onto the sequencing substrate (in red). FIG. 5B depicts, from bottom to top, a substrate (such as silicon, germanium, glass, etc) on which are situated an array of light sources depicted as small purple rectangles (such as microLED, organic LED, quantum dot source, etc). FIG. 5B depicts the leftmost of four light sources emitting light (e.g. ultraviolet light), the second to leftmost light source not emitting light, the third to leftmost light source emitting light, and the rightmost light source not emitting light. FIG. 5B depicts in blue microlenses which collimate and focus the emitted light (e.g. ultraviolet light) onto a sequencing substrate (red). FIG. 5B depicts the microLED-microlens-substrate assembly being brought into contact with an opaque compliant gasket (e.g. silicone gasket, depicted in black), which is then brought into contact with a sequencing substrate. FIG. 5B depicts the opaque compliant gasket as having an array of apertures so as to permit light to travel from the microLED source onto an appropriate matching part of the sequencing substrate while reducing stray light between neighboring and nearby regions of the sequencing substrate. FIG. 5B depicts in red the sequencing substrate containing photocleavable linkers. FIG. 5C illustrates the same assembly as is described by FIG. 5B, but does not show the sequencing substrate, and more clearly illustrates the two dimensional structure of the array of apertures in the opaque gasket and the light source array. FIG. 5C shows the gasket lifted from the light source substrate, but may typically be bonded to the substrate.

FIG. 6 depicts an individual microheater, a microheater array, a microheater array integrated into a sequencing substrate, and a microheater array being selectively turned on or off so as to liberate hybridized strands.

FIG. 7 shows a schematic of another exemplary method for isolation of accurate DNA strands through arrayed substrate-based sequencing (SBS)-based DNA template identification and targeted isolation by UV photocleavage. In this type of SBS. DNA strands and amplicons are immobilized on beads through solid-phase and/or emulsion PCR. Here, the PCR primers immobilized on each bead contain a photocleavable linker. The beads are deposited in a microwell array so that the clonal amplicons on each bead can be sequenced by chemiluminescence-based pyrosequencing (e.g. as commercialized by 454 Life Sciences), fluorogenic pyrosequencing (e.g. as described in Sims et al, Nature Methods, 2011), or using an electronic readout of primer extension (e.g. microscale pH sensing as commercialized by Ion Torrent). In this exemplary embodiment, an SBS method is used to determine the sequence and location of synthesized strands which are immobilized to a substrate with a photopolymer. A targeted illumination machine selectively exposes the substrate in locations corresponding resulting in the cleaving of the photopolymer, followed by a wash, to separate the accurate from inaccurate synthesized strands. A high-accuracy PCR step may be used following the photocleaving process to produce larger volumes of nucleic acid strand product.

FIG. 8 shows a schematic of another exemplary method for isolation of accurate DNA strands. (FIG. 8A) Single-stranded DNA attached to flow cell after sequencing and denaturation. (FIG. 8B) Hybridization of an oligonucleotide containing a photocleavable linker to the 5′-sequencing adapters. (FIG. 8C) Introduction of a nicking enzyme to cleave the 5′-sequencing adapter. (FIG. 8D) DNA attached to flow cell after digestion by nicking enzyme. (FIG. 8E) Selective exposure of desired strands to UV light. (FIG. 8F) DNA attached to flow cell after UV photocleavage of the oligonucleotides hybridized to the desired strands. (FIG. 8G) Dissociation of the desired strands from the flow cell surface. A legend for the figure is also provided.

FIG. 9 is a bar graph showing DNA yield data from an experiment demonstrating targeted photocleavage of nucleic acid strands from a UV-exposed flow cell lane, as shown in FIG. 8, compared to an unexposed lane as measured by fluorometry.

FIG. 10A-10I show another exemplary substrate-based method for isolating desired nucleic acid strands. In this method, the sequence of nucleic acid strands bound to a substrate can be identified, such as by substrate-based sequencing, and each strand can be identified as accurate or inaccurate. An extended sequencing primer is melted from the substrate-bound template strand and washed out of the flow cell (FIG. 10A). A 5-phosphorylated hairpin oligonucleotide containing a photocleavable linker between two nucleotides on its 3′-end is attached to the 3′-ends of all template strands with DNA ligase (FIG. 10B). Next, the 3′-end of the primer is extended with DNA polymerase (FIG. 10D), resulting in replication of the sequenced template strand that is attached to the flow cell (FIG. 10E). The desired strands are selectively exposed to UV light (FIG. 10F), resulting in cleavage of the photocleavable linker in the extended hairpin oligonucleotide (FIG. 10G). Strands are selectively isolated by chemical or thermal melting and extracted from the flow cell (FIG. 10G), resulting in the retention of the undesired strands on the substrate (FIG. 10H). A legend for the above figures is shown in FIG. 10I.

FIG. 11A-11J show another exemplary substrate-based method for isolating desired nucleic acid strands. After sequencing strands attached to a flow cell with substrate-based sequencing (SBS), the extended sequencing primer is melted from the substrate-bound template strand and washed out of the flow cell (FIG. 11A). A 5′-phosphorylated hairpin oligonucleotide containing a photocleavable linker between two nucleotides near its 3′-end and a photoactivatable or photo-reversible terminator at its 3′-end is attached to the 3′-ends of all template strands with DNA ligase (FIG. 11B). Next, the desired strands are selectively exposed to UV light (FIG. 11D), resulting in cleavage of the photocleavable linker and reversion of the photoactivatable or photoreversible terminator to a form that allows primer extension by DNA polymerase. Next, the 3′-end of the primer is extended with DNA polymerase (FIG. 11F), resulting in replication of the sequenced template strand that is attached to the flow cell only for the desired strand (FIG. 11G). Strands are selectively isolated by chemical or thermal melting and extracting the resulting solution from the flow cell (FIG. 11H), resulting in the retention of the undesired strands on the substrate (FIG. 11I). A legend for the above figures is shown in FIG. 11J.

FIG. 12A12L show another exemplary substrate-based method of isolating accurate nucleic acid strands from a mixed library. While clonal amplification of the library strands is used for sequencing, in this example, only the original, desired strands are isolated, rather than a mixture of amplicons of the original, desired strands and/or the original, desired strands. A sample comprising the mixed library is provided to a substrate. The substrate comprises a plurality of nucleic acids bound to distinct locations on the surface of the substrate, without the use of cleavable linkers. The nucleic acid strands are replicated by DNA polymerase, resulting in covalent attachment of their complements to the substrate (FIG. 12A). The nucleic acid strands are then clonally amplified by solid-phase amplification (e.g. bridge PCR or related methods), substituting dUTP for dTTP in the mixture of nucleotides used by DNA polymerase. This results in clonal amplicons containing dU, dA, dG, and dC nucleotide bases, whereas the original strand contains dT, dA, dG, and dC nucleotide bases (FIG. 12B). The sequence of the nucleic acid strands bound to the substrate are identified, and each strand is identified as accurate or inaccurate or as desired or undesired. After sequencing strands attached to a flow cell with substrate-based sequencing (SBS), the extended sequencing primer is melted from the substrate-bound template strand and washed out of the flow cell. A 5″-phosphorylated hairpin oligonucleotide containing a photocleavable linker between two nucleotides on its 3″-end is attached to the 3″-ends of all template strands with DNA ligase (FIG. 12C). Next, a mixture of uracil DNA deglycosylase and endonuclease VIII (i.e. USER enzyme mixture) is introduced, destroying all of the clonal amplification products on the flow cell. The enzyme mixture deglycosylates dU nucleotides, which are absent from the original template strands, and digests strands containing deglycosylated nucleotides (FIG. 12E). Next, the 3′-end of the primer is extended with DNA polymerase (FIG. 12G), resulting in replication of the sequenced template strand that is attached to the flow cell (FIG. 12H). Finally, the desired strands are selectively exposed to UV light (FIG. 12I), resulting in cleavage of the photocleavable linker in the extended hairpin oligonucleotide (FIG. 12J). At this point, the original desired strand can be selectively isolated by chemical or thermal melting and extracting the resulting solution from the flow cell (FIG. 12J), resulting in the retention of the undesired strands on the substrate (FIG. 12K). A legend for the above figures is shown in FIG. 12L.

### DETAILED DESCRIPTION

Section headings as used in this section and the entire disclosure herein are merely for organizational purposes and are not intended to be limiting.

### 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclature used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear: in the event, however of any latent ambiguity, definitions provided herein take precedent over any dictionary or extrinsic definition. Further, unless otherwise required by context, singular terms shall include pluralities and plural terms shall include the singular.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41 (14): 4503-4510 (2002)) and U.S. Pat. No. 5,034,506), locked nucleic acid (LNA: see Wahlestedt et al., Proc. Natl. Acad. Sci. U.S.A., 97:5633-5638 (2000)), cyclohexenyl nucleic acids (see Wang, J. Am. Chem. Soc., 122:8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide.” “nucleotide sequence,” and “oligonucleotide” are used interchangeably. They refer to a polymeric form of nucleotides of any length, either deoxyribonucleotides or ribonucleotides, or analogs thereof.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Systems and Methods for Isolation of Desired Nucleic Acids

The present disclosure provides systems and methods for isolation of nucleic acids. In some embodiments, the disclosure provides systems and methods for isolation of desired nucleic acids.

The systems and methods are used for isolation of desired nucleic acids from a mixed library containing both desired and undesired nucleic acids. A “desired” nucleic acid refers to a nucleic acid strand of which isolation is intended. The term “desired” can refer to either an accurate or an inaccurate nucleic acid strand, depending on the intended isolation strategy. In some embodiments, the “desired” nucleic acid (e.g. the desired nucleic acid to be isolated) is an “accurate” nucleic acid. In such embodiments. “undesired” nucleic acids are “inaccurate” nucleic acids. The term “accurate” is used herein to refer to a nucleic acid having an intended sequence, length, methylation status, modification, or other property. In some embodiments, an “accurate” nucleic acid strand is a nucleic acid strand having an intended sequence. In some embodiments, an “accurate” nucleic acid strand possess another characteristic other than or in addition to an intended sequence. For example, in some embodiments an “accurate” nucleic acid strand is a strand having an intended covalent modification (e.g. covalent DNA modification). For example, an accurate strand may have an intended methylation status. In some embodiments, an “accurate” nucleic acid strand may be a strand that binds or is bound to an intended moiety. For example, an “accurate” nucleic acid strand may bind or be bound to a given protein, such as a fluorescently labeled protein. In other embodiments, the “desired” nucleic acid to be isolated (e.g. isolated from the mixed library containing both desired and undesired nucleic acid strands) is an “inaccurate” nucleic acid. In such embodiments, the “undesired” nucleic acid would be an “accurate” nucleic acid strand. The term “inaccurate” refers to a nucleic acid having one or more mutations or variations that result in the strand not having the intended sequence, length, or other intended property. For example, an “inaccurate” nucleic acid strand may not have the intended sequence. For example, an inaccurate nucleic acid may have one or more substitutions, insertion, or deletion mutations that result in an unintended sequence. As another example, an “inaccurate” nucleic acid strand may not have the correct length. As yet another example, an “inaccurate” strand may not have a desired covalent modification, such as a desired methylation status.

In some embodiments, the systems and methods described herein are used to isolate “desired” nucleic acid strands by performing one or more actions upon accurate strands to isolate them. For example, in some embodiments the methods described herein involve cleaving (e.g. through photocleavage, heat, etc.) accurate strands, thereby releasing them from a substrate, and not cleaving inaccurate strands, thereby allowing them to remain bound to the substrate. Subsequent steps can involve capturing the cleaved accurate strands. However, it is understood that for every method described herein in relation to performing an action on the accurate strands (e.g. allowing them to pass through a nanopore, cleaving them from a substrate) the opposite method is expressly contemplated, wherein an action is performed upon the inaccurate strands rather than the accurate strands. In other words, for every method described herein, the reverse method is expressly contemplated. For example, in some embodiments the “desired” nucleic acid strands to be isolated are inaccurate nucleic acids. Accordingly, in such embodiments the inaccurate nucleic acid strands can be cleaved, such as through photocleavage or application of heat, and subsequently removed from the substrate, thus leaving the accurate strands bound to the surface of the substrate. As another example, in some embodiments the inaccurate strands are permitted passage through a nanopore, whereas the current is modulated through nanopores containing accurate strands, thereby trapping the accurate strands within the nanopore. The inaccurate strands can be removed from system, thus isolating the accurate strands (which remain within the system, and can subsequently be removed after removal of the inaccurate strands).

An “accurate” nucleic acid strand having or determined to have an intended property may refer to a nucleic acid strand having that property with perfect certainty. Alternatively, an “accurate” nucleic acid strand having or determined to have an intended property may refer to a nucleic acid strand having a certain likelihood (e.g. certainty) of having that property. For example, if an “accurate” nucleic acid is determined to “have” a given property, the nucleic acid may have a 1%-100% certainty (or any number therein) that the nucleic acid strand has that property. For example, it may be determined with very high certainty (e.g. at least 95%, at least 96%, at least 97%, at least 98%, at least 99%, at least 99.9%, at least 99.999%, etc.) that a nucleic acid strand has a given property. As another example, it may be determined with at least high certainty (e.g. at least 75%, at least 80%, at least 85%, at least 90%) that a nucleic acid strand has a given property. As another example, it may be determined with at least moderate certainty (e.g. at least 50%, at least 55%, at least 60%, at least 65%, at least 70%) that a nucleic acid strand has a given property. As another example, it may be determined with low certainty (e.g. less than 50% certainty) that a nucleic acid strand has a given property. For example, if an “accurate” nucleic acid strand is determined to “have” a length of 900 bases, there may be some associated uncertainty, such that the nucleic acid is determined to be “accurate” due to a judgment that the length is 90% likely to be between 800 bases and 1000 bases. As another example, if it is determined that a nucleic acid strand is “accurate” because it “has” a specified nucleic acid sequence, the likelihood of the nucleic acid strand containing the specified nucleic acid sequence is may be 1%, 2%, 10%, 25%, 51%, 75%, 80%, 90%, 95%, 99%, 99.9%, 99.999%, or 100%, or any number therein.

An “accurate” strand may have any combination of intended properties, and may approximately or exactly meet any such property or combination of properties. The intended properties and the stringencies for each (e.g. the % certainty of having a given property) may be governed by a system of rules operated by a computer program. The system of rules may be modified (e.g. by a user of the computer program) at any time. For example, an “accurate” strand may be judged to be at least 50% likely (e.g. at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.9% likely) to possess a first intended property or at least 50% likely (e.g. at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.9% likely) to possess a second intended property. As another example, an “accurate” strand may be judged to be at least 50% likely (e.g. at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.9% likely) to possess a first intended property and at least 50% likely (e.g. at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.9% likely) to possess a second intended property. For example, an “accurate” strand may be judged to be at least 50% likely to contain a specific nucleic acid sequence and at least 50% likely to have a length of between 800 and 1200 nucleic acid bases.

|0047| Furthermore, an “accurate” strand may have complex combinations of properties, including but not limited to logical operations, conditionals, control flow, and state dependent on other strands. For example, a nucleic acid strand may be identified as accurate if the length of the strand is between 500 and 600 bases in length, or the strand is between 2000 and 3000 bases in length, or the strand contains a specified nucleic acid sequence with a specified likelihood (e.g. at least 50%) and the strand is between 900 and 1100 bases in length with 99% likelihood. As another example, if any strands in the sample have been detected to contain methylation, then strands containing a specified nucleic acid sequence are accurate, otherwise strands between 3000 and 4000 bases in length are accurate.

The particular intended strand property or combination of intended strand properties may vary by application domain, by application, by experiment within an intended application, over the course of the isolation process in a manner dependent on prior experiments, or data in a shared, remote, or internet-based database. The intended properties may be modified at any given time.

In some embodiments, a decision process is used to select desired/undesired strands. For example, a process used to select strands may incorporate considerations not only of the true positive rate, false positive rate, true negative rate, and false negative rate of the physical strand isolation technique but also considerations of estimates of the error profile of the process used to determine whether a nucleic acid strand is desirable or undesirable in a manner which optimizes the selection process to achieve application-specific goals. Nucleic acid sequencing methods and systems (or other methods and systems which provide information about nucleic acid strands) such as the Illumina NovaSeq provide not only sequencing data such as base sequence determined from a nucleic acid strand as an output, but also may provide an accuracy estimate of that sequence data, such as a per-read quality score. For example, an Illumina NovaSeq sequencing instrument may provide as the data output from a sequencing run, an estimate for each read of a nucleic acid strand on a substrate as having Q20, Q30, Q40, or Q50 accuracy (Phred Score), which are terms of the art referring to 99%, 99.9%, 99.99%, or 99.999% accuracy in the correspondence between the output sequencing data and the actual physical input library nucleotide strand sequence. The term “accuracy” as in “accuracy estimate” from sequence data is a distinct term from “accurate” used in this patent to describe whether or not a nucleic acid strand is considered to be “accurate” as in having an intended property such as an intended sequence. An nucleic acid strand may be determined to have the property of being “accurate” via sequencing data which is of high “accuracy” in the sense of the accuracy estimate or it may be determined to be “accurate” based on sequencing data which is of low “accuracy” in the sense of the accuracy estimate. For example, a nucleic acid strand may be determined to be an “accurate” strand based on sequencing data from that strand which has a Phred Score of Q20, or a nucleic acid strand may be determined to be an “accurate” strand based on sequencing data which has a Phred Score of Q40. The status of whether a nucleic acid strand is considered “accurate” or “inaccurate” is distinct from the accuracy of the information available about that strand, which is, for example, the accuracy of the sequence information available about that strand. Other sequencing methods and systems such as those from Pacific Biosciences. Element Biosciences. Oxford Nanopore, and other current and future manufacturers also often provide information on the expected accuracy of each read or other characteristics of a nucleic acid strand such as methylation. This nucleic acid strand information may be based on a single observation or “raw” read, or it may be the result of repetitious observations of a nucleic acid strand such as Pacific Biosciences HiFi. Oxford Nanopore Duplex. or other multi-pass observations. This accuracy estimate may apply to the entirety of a read, or it may vary along the length of the read. The accuracy estimate provided by a sequencing instrument method or system may also contain more specific information such as an estimate of the likelihood of an insertion or deletion error, the likelihood of a homopolymer error, or an estimate of the likelihood of specific base pair substitutions or the entirety of all possible base pair substitutions, or estimates of the likelihood of errors in the methylation status. The accuracy estimate may be presented as a single number, or it may be presented in a more complex or specific form such as F1 score, precision/recall, or any or all of the true positive rate, false positive rate, true negative rate, and false negative rate. The accuracy estimate may also incorporate information from other sources, such as knowledge of the typical behavior of a sequencing system or method: for example, the accuracy estimate may include knowledge that a sequencing system such as Illumina NovaSeq has an insertion or deletion error rate of approximately two per million. As a specific example scenario of a decision process incorporating this information, an input nucleic acid strand library which is the product of a phosphoramidite synthesis reaction is sequenced via an Illumina NovaSeq sequencing instrument, and a large number of nucleic acid strand reads are made available along with quality score estimates. Any of the above points of information can be used to determine what qualifies as a desired or an undesired nucleic acid strand.

In some embodiments, a user can select which characteristics, including those described above, are to be selected for to isolate desired strands. For example, a user may decide that the accuracy of the nucleic acid strands physically isolated from the input nucleic acid strand library is of paramount importance for a given method, and therefore only nucleic acid strands with reads having both Q40 or higher estimated accuracy and with a perfect sequence identity match may be subsequently physically selected and isolated by methods described herein (e.g. by photocleaving ligated hairpin photolinkers with a two-photon excitation). As an alternative example, a user may decide that only insertion and deletion errors are important for the isolated nucleic acid strands and therefore may opt to ignore substitution errors when selecting and isolating nucleic acid strands and considers strands which are Q20 and above to be desired (e.g. substantially all reads) due to the intrinsic low insertion or deletion error rate of the Illumina NovaSeq method and system.

In some embodiments, the decision process for selecting desired strands incorporates a scoring function, loss function, or probability distribution which provides a mapping between sequence identity or strand characteristics and a numeric value providing an indication of the extent to which the particular sequence identity or strand characteristics will meet the objectives of a particular application. For example, such a scoring function may determine that although a nucleic acid strand has been determined to have a substitution error at a particular location, that the error is not likely to change the corresponding amino acid and therefore determine that the nucleic acid should be considered desirable and should be physically isolated from the substrate. In some embodiments, the decision process optimizes the Kullback-Leibler divergence or cross-entropy loss between a probability distribution, loss function, or scoring function of desired strands and a probability distribution, loss function, or scoring function of observed strands, incorporating information from both or either of the aforementioned accuracy estimate and the aforementioned expectations of the error characteristics of the physical isolation method.

For any of the methods described herein, the method may begin with at least one library containing nucleic acid strands. In some embodiments, the library contains both desired and undesired nucleic acid strands. For example, the mixed library can contain accurate and inaccurate nucleic acid strands. The library containing both desired and undesired strands is referred to herein as a “mixed library”. In some embodiments, the mixed library is a pooled library, containing multiple input libraries. For example, in some embodiments it is advantageous to pool together different input libraries, and then employ multiple isolation steps to isolate desired nucleic acid strands into distinct accurate strand libraries for each of the pooled input libraries. One or more steps may be performed to isolate the desired nucleic acid strands. For example, one or more steps may be performed to isolate the desired nucleic acid strands, thereby generating a library containing only or substantially only accurate nucleic acids. In some embodiments, the nucleic acid is DNA. In some embodiments, the nucleic acid is RNA. In some embodiments, the nucleic acid is single-stranded. In some embodiments, the nucleic acid is double-stranded. In some embodiments, the methods described herein are used to isolate functionalized nucleic acid polymers or highly functionalized nucleic acid polymers.

In some embodiments, the methods described herein comprise isolating a single desired nucleic acid strand. The single desired nucleic acid strand may be single-stranded or double-stranded. In some embodiments, the methods described herein comprise isolating multiple desired nucleic acid strands. The multiple desired nucleic acid strands may be single-stranded or double-stranded. In some embodiments, the multiple desired nucleic acid strands share a common characteristic, such as being part of a clone or a colony with substantially the same sequence. For example, in some embodiments multiple desired nucleic acid strands that are part of a clone or a colony may be isolated for the purpose of amplification for sequencing. In some embodiments, the multiple desired nucleic acid strands that are a part of a clone or a colony are isolated for subsequent sequencing by Illumina sequencing. Solexa sequencing, or Pacific Biosciences sequencing-by-binding.

In some embodiments, a mixed nucleic acid library is subdivided into two nucleic acid libraries, namely the “accurate” and “inaccurate” libraries. In other embodiments, the mixed nucleic acid library may be subdivided into greater than two nucleic acid libraries. For example, one mixed nucleic acid library may be subdivided into three, four, five, ten, one hundred, one thousand, one million, or greater than one million sub-libraries. For example, in some embodiments the original library contains multiple types of strands, and the goal of isolation may be to generate multiple sub-libraries, each sub-library containing a different population of strand types. In such embodiments, strand type “A” may be considered an accurate strand relative to desired feature “A”, but strand type “A” would be considered inaccurate relative to desired feature “B”. Similarly, strand type “B” would be considered an accurate strand relative to desired feature “B”, but would be considered an inaccurate strand relative to desired feature “A”. For example, in some embodiments one mixed nucleic acid library may be subdivided into three or more sub-libraries, wherein each sub-library contains a population of desired nucleic acid strands. For example, sub-library “A” may contain population “A” of desired nucleic acid strands, sub-library “B” may contain population “B” of desired nucleic acid strands, sub-library “C” may contain population “C” of desired nucleic acid strands, etc. In some embodiments, population “A” contains a population of sequences having at least one common desired property, population “B” contains a population of sequences having at least one common desired property that is different from the desired property of population “A” (e.g. a different length, a different sequence, a different methylation status, etc.), and population “C” contains a population of sequences having at least one common desired property that is different from the desired property of population “A” and population “B”.

In some embodiments, the library contains synthetic nucleic acids. In some embodiments, the nucleic acids are synthesized such that a barcode sequence is included (e.g., is contained at one end of the synthesized sequence). The barcode sequence may comprise any suitable number of bases. In some embodiments, the barcode sequence may be used to identify specific subpopulations of intended strands. For example, the methods described herein may be multiplexed, such that multiple nucleic acid strands are intended to be isolated. Multiple unique barcode sequences may be employed to identify the distinct nucleic acid strands intended to be isolated. For example, barcode sequence “A” may be used for intended strand “A”, barcode sequence “B” for intended strand “B”, etc. The barcode sequence may also be used to indicate that the nucleic acid has been completely synthesized. For example, the presence of the barcode sequence indicates that synthesis is complete, whereas the absence of the barcode sequence may indicate an error that resulted in incomplete synthesis of the nucleic acid strand. In some embodiments, the barcode sequence may be cleared and removed following isolation of the intended (e.g. accurate) nucleic acids.

An “isolated nucleic acid strand” may be the original desired strand present in a nucleic acid library, or it may be a complementary strand, such as the nucleic acid strand produced by a polymerase reaction with the original strand.

Isolated nucleic acid molecules (e.g. nucleic acids isolated by the methods described herein) find use in a variety of methods. Isolated nucleic acids may be used, for example, as probes, primers, affinity capture oligonucleotides, guide RNAs (for CRISPR technologies), therapeutic molecules (antisense or RNAi application, gene therapies), aptamers, morpholinos, transcription factor decoys, protein binding molecules, inhibitors, and the like. The nucleic acids may comprise non-natural bases, sugars, and/or backbone modifications. Isolated nucleic acids could also be used as “building blocks” for genomic-scale synthesis. Genomic-scale assembly of such building blocks can enable re-writing of large components of an organism's genetic code. This capability represents an unprecedented opportunity to systematically test the functionality of genomic sequence elements and to impart new capabilities to existing organisms. There are now highly scalable technologies for artificial synthesis of nucleic acid building blocks, but these artificial methods lack the fidelity of naturale DNA synthesis (e.g. with DNA polymerase and the DNA proofreading machinery of the cell). Thus, labor-intensive molecular cloning methodologies are required to isolate accurate nucleic acid building blocks for downstream applications in synthetic biology.

The systems and methods may be used to isolate nucleic acid molecules synthesized, generated, or obtained from any desired source. Such sources include, but are not limited to, phosphoramidite-synthesized nucleic acid, amplified nucleic acids, expressed nucleic acids, affinity captured nucleic acids, purified nucleic acids (e.g., from biological, environmental, or other types of samples), and the like.

### A. Nanopore Sequencing

In some embodiments, provided here in is a system for nanopore sequencing and selective isolation of desired nucleic acid strands. In some embodiments, provided herein is a method for isolation of desired nucleic acids that depends, in part, on nanopore sequencing. As used herein, the term “nanopore sequencing” refers to a sequencing method involving passage of nucleic acids through a nanopore. The nanopore is embedded in a membrane that splits the nanopore sequencing device into two chambers or zones. A difference in electrical potential is generated between the two chambers, such that current passes from one chamber (e.g. the cis chamber) through the nanopore and into the second chamber (e.g. the trans chamber). One or more features of the nucleic acid as it passes through the nanopore may be determined based upon a signal obtained during passage through the nanopore. For example, the sequence, length, and/or covalent modifications present on the nucleic acid as it passes through the nanopore may be determined based upon a signal obtained during passage through the nanopore. In some embodiments, the method comprises determining the sequence of the nucleic acid as it passes through the nanopore. The phrase “determining the sequence” is used herein in the broadest sense and may refer to any process that provides information about one or more properties of the nucleic acid strand. For example, “determining the sequence” of a nucleic acid strand may refer to any sequencing process that determines the nucleotide sequence of a nucleic acid, whether any covalent modifications are present in the nucleic acid (e.g. methylation status), the length of the nucleic acid, whether the nucleic acid is bound to a given entity (e.g. bound to a fluorescent protein), and the like.

In some embodiments, the signal may be an electrical signal. Suitable electrical signals include, for example, current, voltage, tunneling current, resistance, potential, voltage, conductance, and transverse electrical measurements. In some embodiments, disruption of the current flowing through the nanopore may be measured, and decoded to determine whether a given nucleic acid has a desired characteristic (e.g. a desired sequence, length, methylation status, etc.). In some embodiments of nanopore sequencing, passage of the nucleic acid through the nanopore generates a disruption of the current flowing through the nanopore, which can be decoded to determine the sequence of the nucleic acid in real-time, or with a limited time delay, such as one second, one minute, one hour, one day, two days, or up to and including one week. In some embodiments, the method or device involves measuring tunneling current or transverse electron transport (e.g. transverse current). Such sensors and methods are described in technologies marketed by Quantum Biosystems, for example, U.S. Pat. Nos. 9,194,838, 10,202,644, and 10,876,159B2, the entire contents of each of which are incorporated herein by reference for all purposes. In some embodiments, the signal is an optical signal. Suitable optical signals include, for example, a fluorescence signal or a Raman signal. In some embodiments, suitable embodiments of nanopore sequencing include methods based upon optical detection, transverse current detection, hybridization-assisted electrical nanopore detection, and hybridization-assisted fluorescent optical detection.

In some embodiments, the nanopore sequencing device comprises more than two chambers. For example, in some embodiments the device comprises three chambers, wherein the first chamber is separated from the second chamber by a first substantially impermeable membrane, and the second chamber is separated from the third chamber by a second substantially impermeable membrane. The device may comprise any suitable number of chambers. For example, in some embodiments the device comprises more than two chambers such that multiple isolation steps can be performed sequentially.

In some aspects, provided herein is a system for isolation of desired nucleic acid strands. In some embodiments, provided here in is a system for nanopore sequencing and isolation of desired nucleic acid strands. In some embodiments, the system comprises a nanopore sequencing device and a computer that controls one or more operations associated with the nanopore sequencing device. The nanopore sequencing devices comprises at least two chambers or zones. In some embodiments, the chambers or zones are separated by a substantially impermeable membrane. In some embodiments, multiple substantially impermeable membranes are present (e.g. a first membrane in between a first and second chamber, a second membrane between a second and third chamber, a third membrane in between a third and fourth chamber, etc.) The term “substantially impermeable” indicates that the membrane is impermeable to passage of nucleic acids, except for through the nanopores embedded within the membrane. Any suitable membrane may be used in the systems and methods described herein. For example, suitable membranes are described in International Application No. WO2021/111125. International Application No. WO2014/064443, and WO2014/064444, the entire contents of each of which are incorporated herein by reference for all purposes.

In some embodiments, the substantially impermeable membrane is an amphiphilic layer formed from amphiphilic molecules, such as phospholipids, which have both hydrophilic and lipophilic properties. The amphiphilic molecules may be synthetic or naturally occurring. Non-naturally occurring amphiphiles and amphiphiles which form a monolayer are known in the art and include, for example, block copolymers (Gonzalez-Perez et al. Langmuir. 2009, 25, 10447-10450, the entire contents of which are incorporated herein by reference for all purposes). Block copolymers are polymeric materials in which two or more monomer sub-units that are polymerized together to create a single polymer chain. In some embodiments, block copolymers are engineered such that one of the monomer sub-units is hydrophobic (i.e. lipophilic), whilst the other subunits) are hydrophilic whilst in aqueous media. Accordingly, the block copolymer may possess amphiphilic properties and may form a structure that mimics a biological membrane. The block copolymer may be a diblock (e.g. consisting of two monomer subunits). In other embodiments, the block copolymer may be constructed from more than two monomer sub-units to form more complex arrangements that behave as amphipiles. For example, the copolymer may be a triblock, tetrablock or pentablock copolymer.

In some embodiments, a block copolymer material may be constructed to mimic archaebacterial bipolar tetraether lipids. Archaebacterial bipolar tetraether lipids are naturally occurring lipids that are constructed such that the lipid forms a monolayer membrane. These lipids are generally found in extremophiles that survive in harsh biological environments, thermophiles, halophiles and acidophiles, and are therefore highly stable. In some embodiments, a block copolymer material may be constructed to mimic archaebacterial bipolar tetraether lipids, such as a triblock polymer that has the general motif of hydrophilic-hydrophobic-hydrophilic. In some embodiments, block copolymers may be synthesized to provide the correct chain lengths and properties required to form membranes and to interact with pores and other proteins.

Block copolymers may also be constructed from sub-units that are not classed as lipid sub-materials: for example a hydrophobic polymer may be made from siloxane or other non-hydrocarbon based monomers. In some embodiments, block copolymer membranes have increased mechanical and environmental stability compared with biological lipid membranes, for example a much higher operational temperature or pH range, and therefore provide a highly flexible synthetic solution for use in the systems and methods described herein.

In some embodiments, the substantially impermeable membrane is a lipid bilayer. The lipid bilayer may be any lipid bilayer. Suitable lipid bilayers include, but are not limited to, a planar lipid bilayer, a supported bilayer or a liposome. In some embodiments, the lipid bilayer is a planar lipid bilayer. Suitable lipid bilayers are disclosed in International Application No. WO 2008/102121, International Application No. WO 2009/077734, and International Application No. WO 2006/100484, the entire contents of each which are incorporated herein by reference for all purposes.

Generally speaking, a lipid bilayer is formed from two opposing layers of lipids. The two layers of lipids are arranged such that their hydrophobic tail groups face towards each other to form a hydrophobic interior. The hydrophilic head groups of the lipids face outwards towards the aqueous environment on each side of the bilayer. The bilayer may be present in a number of lipid phases including, but not limited to, the liquid disordered phase (fluid lamellar), liquid ordered phase, solid ordered phase (lamellar gel phase, interdigitated gel phase) and planar bilayer crystals (lamellar sub-gel phase, lamellar crystalline phase). The lipids may comprise naturally-occurring lipids and/or artificial lipids.

The lipids typically comprise a head group, an interfacial moiety and two hydrophobic tail groups which may be the same or different. Suitable head groups include, but are not limited to, neutral head groups, such as diacylglycerides (DG) and ceramides (CM): zwitterionic head groups, such as phosphatidylcholine (PC), phosphatidylethanolamine (PE) and sphingomyelin (SM); negatively charged head groups, such as phosphatidylglycerol (PG): phosphatidylserine (PS), phosphatidylinositol (PI), phosphatic acid (PA) and cardiolipin (CA); and positively charged headgroups, such as trimethylammonium-Propane (TAP). Suitable interfacial moieties include, but are not limited to, naturally-occurring interfacial moieties, such as glycerol-based or ceramide-based moieties. Suitable hydrophobic tail groups include, but are not limited to, saturated hydrocarbon chains (e.g. lauric acid, myristic acid, palmitic acid, stearic acid, and arachidic acid), unsaturated hydrocarbon chains (e.g. oleic acid); and branched hydrocarbon chains (e.g. phytanoyl). In some embodiments, the lipids may be chemically modified. The lipid bilayer may comprise one or more additives to influence the properties of the layer.

In some embodiments, the membrane is a solid-state (e.g. synthetic) membrane. Solid state membranes may be formed from both organic and inorganic materials including, but not limited to, microelectronic materials, insulating materials such as S13N4. AI2O3, and SiO, organic and inorganic polymers such as polyamide, plastics such as Teflon® or elastomers such as two-component addition-cure silicone rubber, and glasses. In some embodiments, the solid state membrane may be formed from graphene. Suitable graphene layers are disclosed in International Application No. WO 2009/035647, the entire contents of which are incorporated herein by reference. In some embodiments, the solid state membrane is a silicon based membrane. Suitable silicon based membranes include, for example. SiNx or SiO₂ membranes. In some embodiments, the membrane is electro-resistant.

In some embodiments, the nanopore sequencing device comprises at least one nanopore. In some embodiments, the nanopore sequencing device comprises at least one nanopore embedded within the substantially impermeable membrane. In some embodiments, the nanopore sequencing device comprises a plurality of nanopores embedded within the substantially impermeable membrane. As used herein, the term “nanopore” refers to any opening positioned in a substrate (e.g. in the substantially impermeable membrane) that allows the passage of analytes through the substrate (e.g. through the membrane) in a discernable order. In the case of nucleic acids, the nanopore permits passage of the monomeric units (e.g. nucleotide or ribonucleotide bases) through the membrane in a discernable order.

A wide variety of nanopores and substantially impermeable membranes comprising the same may be used to achieve the intended sequencing in the methods described herein. Suitable nanopores, including biological nanopores and membranes comprising the same are reviewed in Feng et al., Genomics Proteomics Bioinformatics. 2015 Feb.; 13 (1): 4-16, the entire contents of which are incorporated herein by reference for all purposes. Suitable nanopores and membranes comprising the same are additionally described in, for example. International Application No. WO/2021/111125, the entire contents of which are incorporated herein by reference for all purposes. The nanopores may be biological nanopores. In some embodiments, the nanopore may be a protein nanopore, a synthetic or solid state nanopore, or a hybrid nanopore.

In some embodiments, the nanopore is a protein nanopore. Examples of protein nanopores include, but are not limited to, alpha-hemolysin, anthrax toxin, leukocidins, lysenin, ClyA, spl, haemolytic protein fragaceatoxin C (FraC), voltage-dependent mitochondrial porin (VDAC), OmpF, OmpG, NaIP, OmpC, MspA, MspB, MspC, MspD, CsgG, and LamB (maltoporin). For example, the nanopore may be an α-hemolysin nanopore. A-hemolysin nanopores have an inner diameter of about 1 nm, which may be particularly well suited for passage of DNA through the nanopore. Accordingly, suitable α-hemolysin nanopores may be useful to discriminate ionic current at the single nucleotide level (see. e.g., Cherf G., Lieberman K., Rashid H., Lam C., Karplus K., Akeson M. Automated forward and reverse ratcheting of DNA in a nanopore at 5-a precision. Nat Biotechnol. 2012:30:344-348, the entire contents of which are incorporated herein by reference). As another example, the nanopore may be an MsPA nanopore, which has been successfully used for improved spatial resolution of single-stranded DNA sequencing (Laszlo A. H., Derrington I. M., Ross B. C., Brinkerhoff H., Adey A., Nova I. C. Decoding long nanopore sequencing reads of natural DNA. Nat Biotechnol. 2014:32:829-833, the entire contents of which are incorporated herein by reference). In some embodiments, the biological nanopore may be bacteriophage phi29 (i.e. phi29), which may be particularly useful for applications using larger molecules such as double stranded DNA (Haque F., Guo P. Membrane-embedded channel of bacteriophage phi29 DNA-packaging motor for translocation and sensing of double-stranded DNA. In: Iqbal S. M., Bashir R., editors. Nanopores. Springer US: New York: 2011. pp. 77-106, Wendell D., Jing P., Geng J., Subramaniam V., Lee T. J., Montemagno C. Translocation of double-stranded DNA through membrane-adapted phi29 motor protein nanopores. Nat Nanotechnol. 2009:4:765-772, the entire contents of each of which are incorporated herein by reference).

In some embodiments, the nanopore may be adapted to modify the architecture of the internal structure of the nanopore, such as to accommodate specific desired nucleic acids. As another example, the nanopore may be functionalized with a DNA probe, a molecular motor, and/or various ligands/aptamers, which may be used to bind with target proteins outside of the pore. For example, the nanopore may be functionalized to be particularly well suited for binding and subsequent transport of a given nucleic acid target. In some embodiments, the nanopore is a protein pore comprising one or more mutations compared to the wildtype protein. Suitable mutant pores are described in, for example, U.S. Pat. Nos. 10,167,503, 10,995,372, 10,975,428, 9,751,915, 9,777,049, 10,882,889, 10,400,014, 11,034,734, and International Application No. WO/2020/208357A1, the entire contents of each of which are incorporated herein by reference for all purposes.

Alternatively, the nanopores may be synthetic nanopores. Synthetic nanopores are also referred to herein as solid-state or solid state nanopores. In some embodiments, the nanopore is a solid-state nanopore (e.g. a pore formed in a synthetic solid-state membrane, such as an SiNx or SiO₂ membrane). In some embodiments, the nanopore is a solid-state nanopore formed in a membrane comprising silicones, metals, metal oxides, plastics, glass, semiconductor materials, or combinations thereof. In some embodiments, synthetic nanopores are more stable than biological nanopores positioned in a lipid bilayer membrane. In some embodiments, the nanopore is a graphene nanopore (e.g. a nanopore formed within a graphene membrane). In some embodiments, the nanopore is a hybrid pore (e.g. a solid state nanopore having a protein nanopore embedded therein). In some embodiments, the nanopore is a glass micropipette/nanopipette nanopore, a boron-nitride nanopore, or a silicon-stabilized graphene nanopore.

In some cases, the nanopore can be a solid state nanopore. A review of suitable solid state nanopores and membranes, along with suitable methods of creating the same, is disclosed in Fried et al., Chem Soc Rev. 2021 Apr. 26; 50 (8): 4974-4992, the entire contents of which is incorporated herein by reference for all purposes. Suitable solid state nanopores are described in, for example. Storm. A. J., Chen. J. H., Ling. X. S., Zandbergen. H. W. & Dekker. C. Fabrication of solid-state nanopores with single nanometre precision. Nature Mater. 2, 537-540) (2003): Venkatesan. B. M. et al. Highly sensitive, mechanically stable nanopore sensors for DNA analysis. Adv. Mater. 21, 2771-2776 (2009): Kim. M. J., Wanunu. M., Bell. D. C. & Meller. A. Rapid fabrication of uniformly sized nanopores and nanopore arrays for parallel DNA analysis. Adv. Mater. 18, 3149-3153 (2006): Nam. S-W., Rooks. M. J., Kim. K-B. & Rossnagel. S. M. Ionic field effect transistors with sub-10 nm multiple nanopores. Nano Lett. 9, 2044-2048 (2009); Healy. K., Schiedt. B. & Morrison. A. P. Solid-state nanopore technologies for nanopore-based DNA analysis. Nanomedicine 2, 875-897 (2007); U.S. Pat. Nos. 7.258.838; and U.S. Pat. No. 7,504,058, the entire contents of which are incorporated herein by reference for all purposes.

In some cases, graphene can be used, as described in: Geim. A. K. Graphene: status and prospects. Science 324, 1530-1534 (2009): Fischbein, M. D. & Drndic. M. Electron beam nanosculpting of suspended graphene sheets. Appl. Phys. Lett. 93, 113107-113103 (2008); Girit. Ç. Ö. et al. Graphene at the edge: stability and dynamics. Science 323, 1705-1708 (2009): Garaj. S. et al. Graphene as a subnanometre trans-electrode membrane. Nature 467, 190-193 (2010): 52. Merchant. C. A. et al. DNA translocation through graphene nanopores. Nano Lett. 10, 2915-2921 (2010): Schneider. G. F. et al. DNA translocation through graphene nanopores. Nano Lett. 10, 3163-3167 (2010): Hall. J. E. Access resistance of a small circular pore. J. Gen. Physiol 66, 531-532 (1975); and Song. B. et al. Atomic-scale electron-beam sculpting of near-defect-free graphene nanostructures. Nano Lett. 11, 2247-2250 (2011), each of which are incorporated herein by reference in their entirety for all purposes. Suitable graphene layers and nanopores within the same are additionally described in International Application No. WO/2009/035647, which is incorporated herein by reference in its entirety.

In some cases the nanopore comprises a hybrid protein/solid state nanopore in which a nanopore protein is incorporated into a solid state nanopore. Suitable nanopores are described, for example in Mager. M. D. & Melosh. N. A. Nanopore-spanning lipid bilayers for controlled chemical release. Adv. Mater. 20, 4423-4427 (2008): White. R. J. et al. Ionic conductivity of the aqueous layer separating a lipid bilayer membrane and a glass support. Langmuir 22, 10777-10783 (2006); Venkatesan. B. M. et al. Lipid bilayer coated Al₂O₃ nanopore sensors: towards a hybrid biological solid-state nanopore. Biomed. Microdevices 13, 671-682 (2011) which are incorporated herein by reference in their entirety for all purposes. Additional hybrid nanopores are described, for example, in U.S. Publication No. 2010/0331194: Iqbal. S. M., Akin. D. & Bashir. R. Solid-state nanopore channels with DNA selectivity. Nature Nanotech. 2, 243-248 (2007); Wanunu. M. & Meller. A. Chemically modified solid-state nanopores. Nano Lett. 7, 1580-1585 (2007); Siwy. Z. S. & Howorka. S. Engineered voltage-responsive nanopores. Chem. Soc. Rev. 39, 1115-1132 (2009); Kowalczyk. S. W. et al. Single-molecule transport across an individual biomimetic nuclear pore complex. Nature Nanotech. 6, 433-438 (2011): Yusko. E. C. et al. Controlling protein translocation through nanopores with bio-inspired fluid walls. Nature Nanotech. 6, 253-260 (2011): Bai J. W., Wang D. Q., Nam S. W., Peng H. B., Bruce R., Gignac L. Fabrication of sub-20 nm nanopore arrays in membranes with embedded metal electrodes at wafer scales. Nanoscale. 2014:6:8900-8906; and Hall. A. R. et al. Hybrid pore formation by directed insertion of alpha-haemolysin into solid-state nanopores. Nature Nanotech. 5, 874-877 (2010), each of which are incorporated herein by reference in their entirety for all purposes.

The nanopore may be any desired shape or dimensions. In some embodiments, the nanopore has an inner diameter of about 1-10 nm. For example, the nanopore may have an inner diameter of about 1 nm, about 2 nm, about 3 nm, about 4 nm, about 5 nm, about 6 nm, about 7 nm, about 8 nm, about 9 nm, or about 10 nm. In some embodiments, the nanopore may be selected and optimized based upon the accurate (e.g. desired) sequence of the nucleic acid. For example, the nanopore may be optimized to facilitate passage of the desired nucleic acid through the nanopore while preventing passage of undesired contaminants through the pore.

In some embodiments, the plurality of nanopores are arranged in an array. In some embodiments, the nanopore sequencing device comprises an array of microscaffolds, wherein each microscaffold supports a membrane containing the embedded nanopore. In such embodiments, the array of microscaffolds are considered a part of the “substantially impermeable membrane”. In other words, the “substantially impermeable membrane” comprises the array of microscaffolds. Accordingly, each microscaffold supports a single electrode, and the “substantially impermeable membrane” comprising the plurality of microscaffolds therefore comprises a plurality of nanopores housed within the membrane. In some embodiments, the device further comprises a plurality of electrodes. In some embodiments, each microscaffold (e.g. each microscaffold, which supports each embedded nanopore) may be controlled by its own electrode. In some embodiments, each electrode is connected to a distinct channel, such that the voltage applied through each electrode may be independently controlled. Accordingly, the current passing through each individual nanopore may also be independently controlled.

In some embodiments, each nanopore within the array is substantially identical. In other embodiments, multiple types of nanopores are used. For example, in some embodiments it may be advantageous to employ different nanopores for sequencing of multiple nucleic acids. For example, one nanopore may be advantageous for a nucleic acid having the intended sequence A, another nanopore may be advantageous for a nucleic acid having the intended sequence B, etc. In some embodiments, they system comprise additional chambers or zones associated with particular, different nanopores such that accurate nucleic acid molecules of particular types are physically segregated from one another and from inaccurate nucleic acid molecules.

In some embodiments, the device further comprises a plurality of sensors. The sensors detect a signal which can be decoded to determine the sequence of the nucleic acid passing through a given nanopore. Suitable sensors and types of signals that can be detected are described in, for example, U.S. Pat. Nos. 11,041,196, 10,364,462, and 9,689,033, the entire contents of each of which are incorporated herein by reference for all purposes. In some embodiments, the signal is an electrical signal. Suitable sensors and types of signals are also described in the work of Gundlach et al. e.g. U.S. Pat. No. 9,588,079B2. Accordingly, in some embodiments the sensor detects an electrical signal as the nucleic acid strand passes through a given nanopore. Suitable electrical signals include, for example, current, voltage, tunneling, resistance, potential, voltage, conductance, and transverse electrical measurements. In some embodiments, the device comprises a plurality of sensors to record the current passing through each nanopore, which can be decoded to identify the sequence of the base within the nanopore. The presence of a given nucleotide base (e.g. adenine (A), guanine (G), thymine (T), cytosine (C), uracil (U), or synthetic variants thereof) will generate a characteristic disruption in the current passing through the nanopore, thus facilitating sequencing of the strand as it passes through the nanopore. In other words. A. G. T. C, and U each generate an identifiably disruption in the current, and therefore each base pair can be identified as it passes through the nanopore. The sensors may be placed at a suitable location along the channels, such that a plurality of sensors are arranged in an array (e.g., an array corresponding to the locations of the channels controlling the flow of current through each nanopore).

In some embodiments, the sensors are optical sensors. For example, in some embodiments the device further comprise one or more optical sensors that detect a label (e.g. a fluorescent moiety or a Raman signal generating moiety) on the nucleic acid strand. In some embodiments, the optical signal is then used to determine the nucleotide sequence of the strand passing through a given nanopore. Suitable methods for optical signal based nanopore sequencing methods are described in, for example. Son et all. Rev Sci Instrum 2010; 81 (1): 014301: McNally et al., Nano Lett. 2010; 10 (6): 2237-2244; U.S. Pat. No. 10,823,721. U.S. Pat. No. 9,862,997. U.S. Pat. No. 10,597,712. U.S. Patent Publication No. 2019/0112649, and U.S. Patent Publication No. 2019/0078158, the entire contents of each of which are incorporated herein by reference for all purposes.

In some embodiments, the system further comprises a computer. The computer may be operably connected to one or more components of the nanopore sequencing device. For example, the computer may be operably connected to the electrodes to control the voltage applied to each channel. The computer may be operably connected to the sensors. For example, the computer may be operably connected to the sensors to receive a reading of the current passing through a given nanopore. As another example, the computer may be operably connected to the sensors to receive a reading of an optical signal detected by the sensors as the nucleic acid strand passes through a given nanopore. The computer may comprise a memory and a processor, wherein the memory encodes instructions that dictate that the processor perform a given task. In some embodiments, the computer employs an algorithm to determine the sequence of nucleic acid strands passing through the nanopore based upon the signal detected by the sensors. For example, the sequence may be determined based upon the optical signal detected by the sensors. As another example, the sequence may be determined based upon the electrical signal detected by the sensors. In some embodiments, the computer employs an algorithm to determine the sequence of nucleic acid strands passing through a given nanopore based upon the characteristic changes in current that indicate a given nucleobase or variant thereof is present in the nanopore. The algorithm may additionally compare the sequence of a given nucleic acid strand to the intended sequence to determine whether one or more mutations are present in a given nucleic acid strand. The algorithm may be encoded in software, which may be stored in a memory of the computer. Alternatively, the algorithm may be encoded in hardware, which may be operably connected to the computer prior to use (e.g. inserted as a CD-ROM, external disc, external hard drive, etc.).

In some embodiments, the system comprises software. In some embodiments, the software is stored on a computer. For example, the software may be stored in a memory of the computer. In some embodiments, the software may be stored on an external medium, such as a CD-ROM, a floppy disk, a hard drive, a DVD, a Blu-ray disk, a solid-state storage media such as a flash solid-state storage media, etc., which may be suitably connected to the computer prior to executing the software stored therein. In some embodiments, the software is designed to execute one or more tasks in a method of nanopore sequencing as described herein. In some embodiments, the software instructs a processor to execute a given task. In some embodiments, the software stores machine readable instructions. For example, in some embodiments the software stores machine readable instructions that instruct the processor to execute a given task. The machine readable instructions may be one or more executable programs or portion(s) of an executable program for execution by a processor.

In some embodiments, the software collects and analyzes data from the nanopore sequencing device. For example, in some embodiments the software collects and analyzes data regarding the sequences or length or other properties of nucleic acid strands passing through the nanopores in the nanopore sequencing device. In some embodiments, the software encodes an algorithm which is employed to determine the sequence of a given nucleic acid strand passing through a nanopore based upon the signal (e.g. optical or electrical signal) detected by the one or more sensors. In some embodiments, the algorithm determines the sequence of a given nucleic acid based upon characteristic changes in current that indicate a given nucleobase or variant thereof is present in the nanopore. In some embodiments, the software analyzes the sequence data, such as by comparing the sequence of a given nucleic acid strand to the sequence of a desired (e.g. accurate) nucleic acid strand.

In some embodiments, the software actuates other components of the system to control the isolation of desired strands from undesired strands. For example, the software may instruct the processor to perform one or more functions, thereby controlling isolation of desired strands from undesired strands. For example, the software may control the voltage applied to each channel via the electrodes of the nanopore sequencing device. The software may instruct the processor to modulate the voltage at a given channel depending on the sequence of a nucleic acid passing through the nanopore of that channel, thereby controlling flux of the nucleic acid strand through the nanopore. Accordingly, the software may instruct the processor to modulate the voltage at a given channel to selectively release either a desired or an undesired nucleic acid strand from a given nanopore. For example, the software may dictate that the voltage of a given electrode is not modified when the nucleic acid strand passing through the nanopore operably connected to said electrode is desired (e.g. accurate). Alternatively, the software may dictate that the voltage of a given electrode is modified to cease or reverse the flow of current through a nanopore operably connected to said electrode when the nucleic acid strand passing through the nanopore is undesired (e.g. inaccurate). For example, in some embodiments the voltage is reversed, such that the strand passing through the nanopore is ejected.

In some embodiments, the computer operates autonomously. For example, a user may provide a set of instructions to the computer, and the computer may perform tasks in accordance with said instructions autonomously. In other embodiments, the computer does not operate autonomously. For example, during one or more steps performed by the computer the computer may prompt the user for input. The user may provide said input to the computer. and based upon the user's input the computer may perform a given task.

In some aspects, provided herein are methods of isolating desired nucleic acid strands. The methods may be performed using a system as described herein. In some embodiments, the methods comprise obtaining a mixed library containing both desired and undesired nucleic acids. The library may be transferred to a first chamber (e.g. the cis chamber) of a nanopore sequencing device. A difference in electrical potential between two chambers (e.g. between a cis chamber and a trans chamber) may be generated, such as by applying a voltage to the cis chamber, such that nucleic acid strands begin the process of translocating through the nanopore. During translocation, disruption of the current through the nanopore may be used to determine whether a given nucleic acid passing through the nanopore has a desired feature (e.g. a desired sequence, a desired length, a desired methylation status, a desired protein-binding status, etc.). In some embodiments, disruption of the current is measured in real-time. In some embodiments, disruption of the current is measured and decoded to determine the sequence of the nucleic acid. Accordingly, deviations from the desired sequence are identified in real-time. In some embodiments, undesired strands may be driven back into the cis chamber and/or held within the nanopore, whereas desired strands may be permitted to pass through the nanopore and into the trans chamber. Desired nucleic acid sequences may then be collected. In some embodiments, nucleic acids may be collected from the trans chamber. In some embodiments, nucleic acids may be collected from the cis chamber. In some embodiments, the “desired” strands are accurate nucleic acid strands. Accordingly, in some embodiments the accurate nucleic acid strands are permitted to pass through the nanopore and into the trans chamber, whereas inaccurate nucleic acid strands are halted and/or driven back. In other embodiments, the “desired” strands are inaccurate nucleic acid strands. In such embodiments, the accurate strands are driven back into the cis chamber and/or held within the nanopore, whereas the inaccurate strands are permitted to pass through the nanopore and into the trans chamber. In some embodiments, the inaccurate strands are removed from the trans chamber, thus leaving behind the accurate strands. In some embodiments, the accurate strands are then collected, such as by permitting them to pass into the trans chamber or reversing the current and ejecting the accurate strands back into the cis chamber, followed by isolating the strands.

In some embodiments, multiple isolation steps are performed, such as to increase accuracy of separation. For example, in some embodiments a first isolation step may be performed to obtain a first population containing desired nucleic acids. The first population may be submitted to a second round of purification, either by adding the first population to the first chamber and passing through nanopores a second time, or by passing the first population through a second semi-impermeable within the nanopore sequencing device. Such multiple purifications may further enrich a given population of desired nucleic acids and/or help increase accuracy of purification (e.g. further eliminate undesired strands) through additional purification steps.

In some embodiments, a computerized process is used to identify deviations from the desired strand, and to determine whether a given strand should be permitted passage through the nanopore. The term “computerized” as used herein refers to a process performed using a computer. For example, in some embodiments a computerized process is used to compare the sequence of the nucleic acid strand passing through the nanopore to the intended sequence of the strand. Desired nucleic acid strands may be permitted to pass completely through the nanopore. For example, accurate nucleic acid strands having the intended sequence may be permitted to pass completely through the nanopore, whereas nucleic acid strands containing one or more mutations, length differences, or other undesired properties from the expected sequence (e.g., inaccurate nucleic acids) may be prevented from passing through the nanopore. For example, the channel controlling the passage of current through the nanopore containing the inaccurate nucleic acid strand may be controlled by the computer, such that the applied voltage is modified to reduce the flow of current through the nanopore. Accordingly, the passage of the nucleic acid strand through the nanopore may be halted, such as immediately after identification of a single mutation or after identification of multiple mutations.

In some embodiments, the inaccurate nucleic acid strands may be contained within the nanopores whereas accurate nucleic strands are permitted passage to the trans chamber. In some embodiments, accurate nucleic acid strands are isolated directly from the trans chamber. In some embodiments, the inaccurate nucleic acid strands may be ejected from the nanopore and back into the cis chamber. For example, the applied voltage may be modified such that the flow of current is reversed (e.g. current flows from the nanopore back into the cis chamber), thereby ejecting the inaccurate nucleic acid stands. In still other embodiments, accurate nucleic acid strands may be passed from the chamber to a third chamber. For example, the device may comprise a first chamber and a second chamber separated by a first substantially impermeable membrane. Accurate nucleic acid strands may be permitted passage through the nanopores into the second chamber. Following passage, a second voltage may be applied to the nanopores embedded within a second substantially impermeable membrane that separates the second chamber from a third chamber. The sequence of nucleic acids passing through the nanopores within the second substantially impermeable membrane may be determined, and accurate nucleic acids may be granted complete passage into the third chamber. Such embodiments may also be useful for enriching a low-abundance population of nucleic acids. Such embodiments may also be useful for generating distinct populations of nucleic acids of interest. For example, the nucleic acids of sequence “A” may be held within the first chamber (e.g. not permitted passage through the nanopores in a first substantially impermeable membrane, whereas nucleic acids of sequence “B” and sequence “C” may be permitted passage through the first substantially impermeable membrane into the second chamber. Nucleic acids of sequence “B” may be held in the second chamber, whereas nucleic acids of sequence “C” may be permitted passage into the third chamber (e.g. allowed to translocate through the nanopores embedded within a second impermeable membrane separating the second and third chambers). The separate populations of nucleic acids may then be isolated and further amplified, if desired.

In some embodiments, upon passage of the accurate nucleic acid strands through the nanopore and into the trans chamber and containment of the inaccurate nucleic acid strands within the cis chamber, the inaccurate nucleic acid strands may be removed. For example, the cis chamber containing the inaccurate nucleic acid strands may be evacuated (e.g. aspirated). In some embodiments, one or more wash steps may be performed to further remove unwanted nucleic acid strands from the cis chamber.

In some embodiments, after removal of the inaccurate nucleic acid strands (and optionally the one or more wash steps, if performed) the flow of current may be reversed again such that all accurate nucleic acid strands held within the trans chamber pass through the nanopore and back into the cis chamber. Accordingly, the method results in a library of accurate nucleic strands held within the cis chamber, which may be readily aspirated or otherwise obtained and used for the desired purpose.

In some embodiments, the computer stores instructions that facilitate proper execution of multiple processes performed using the methods as described herein. For example, the computer may store instructions that instruct the computer to regulate the voltage applied to the channels, record the current passing through each nanopore, determine the sequence of the nucleic acid strand passing through each nanopore, compare the sequence of each nucleic acid strand to the intended sequence, and modulate the voltage applied to each channel as necessary. In some embodiments, the computer executes a decision-tree algorithm to determine whether to modulate the voltage applied to each channel. For example, the computer may execute a decision-tree algorithm that determines whether to permit passage of the nucleic acid strand through the nanopore, or whether to modulate the voltage (e.g. to stop the flow of current and trap the nucleic acid strand within the nanopore, to reverse the flow of current through the nanopore to “eject” the nucleic acid strand, etc.). In some embodiments, the decision-tree algorithm dictates that a single mutation (e.g. a single point mutation such a base substitution, deletion, or insertion) is sufficient to cease the flow of current through the nanopore and trap the nucleic acid strand within the nanopore.

In some embodiments, the passage of the nucleic acid strand through the nanopore occurs in only one direction and only once, with no reversal of the direction of passage or alteration in speed. In other embodiments, the translocation of the nucleic acid strand occurs in both a forward and reverse direction any number of times, so as to gain more information about the nucleic acid strand or to gain redundant information about the nucleic acid strand. In some embodiments an alternating current is used to improve the accuracy of determination of properties of the nucleic acid strand such as its sequence, length, methylation status, or other (Noakes MT. Brinkerhoff H. Laszlo AH, et al. Increasing the accuracy of nanopore DNA sequencing using a time-varying cross membrane voltage. Nat Biotechnol. 2019: 37 (6): 651-656. doi: 10.1038/s41587-019-0096-0).

In some embodiments, the method is multiplexed. For example, multiple desired strands may be isolated using the methods described herein. For example, accurate strand “A”, accurate strand “B”, and accurate strand “C” may each be present within the initial mixed library along with inaccurate strands for each. The computerized process may involve a step of determining which strand is passing through a given nanopore, and comparing that strand to the accurate strand for the appropriate nucleic acid (e.g. nucleic acid having the accurate strand “A”, “B”, or “C”, for example.).

In some embodiments, the computerized process may be used for de-multiplexing, to generate selective libraries containing subpopulations of useful nucleic acid strands. For example, the computerized process may be used to modulate the voltage in a specific subset of channels, such that the flow of current through nanopores containing a subpopulation of nucleic acids is reversed. The subpopulation may be collected. Subsequently, the voltage in another subset of channels may be modulated such that the flow of current through nanopores containing a second subpopulation of nucleic acids is reversed. This second subpopulation may be collected. The process may be repeated as needed to achieve the intended de-multiplexing.

In some embodiments, a nanopore is used to determine the characteristics of a strand in order to identify whether the strand is desired or not desired, and a method other than or in addition to changing the current through the nanopore is utilized in order to selectively isolate the desired strand(s). For example, in some embodiments the nucleic acid strands are ligated to a linker (e.g. a photolinker, a heat-sensitive linker). The linker may serve to anchor the nucleic acid strand to a substrate, such as to a lipid bilayer or to a bead, the linker may join the nucleic acid strand to a strand designed for capture by hybridization, or the linker may link the nucleic acid strand to a primer. In some embodiments, the characteristics (e.g. sequence) of a strand are determined as the strand passes through a nanopore, and selective cleavage of the linkers attached to desired strands is induced to release the desired strands from the nanopore, while containing undesired strands within the nanopore. Suitable methods for cleaving the linkers are described herein, and include selective application of a light stimulus (e.g. UV, one-photon, two-photon, three-photon, or other multi-photon) or heat stimulus to the desired area, thereby selectively releasing the desired strands from the nanopore. The desired strands can be isolated, such as by washing. In some embodiments, the current through the nanopore can be reversed following selective release of the desired strands, thereby ejecting the undesired strands back into the other chamber. If the nucleic acid strand is ligated to a linker which anchors the nucleic acid strand to a substrate, such as to a lipid bilayer or to a bead, the cleaving of the linker frees the desired strand which may then be isolated by a wash step separating the desired strand from the substrate. If the nucleic acid strand is ligated to a linker which attaches the nucleic acid strand to a capture strand, the undesired stands may be separated from the desired strands by hybridizing the nucleic acid strands to capture probes which are bound to a substrate, such as capture beads followed by a wash—in this circumstance the nucleic acid strands which are be bound to the capture bead will be separated from strands which are washed away. If the desired nucleic acid strands are linked to a primer, a PCR amplification step may be applied to amplify the nucleic acid strands which are linked to a primer, and not amplify nucleic acid strands which have had their primer cleaved.

In some embodiments, the isolated nucleic acid strands may be further amplified. Suitable amplification techniques include polymerase chain reaction (PCR) and variants thereof. Such amplification methods may be used to increase the number of strands within the library of accurate nucleic acids.

The nucleic acids isolated by the methods described herein (e.g. the desired nucleic acids) may be used for a variety of purposes. In some embodiments, the isolated nucleic acids ay be used for targeting sequencing. For example, performing the nanopore-guided methods described herein followed by targeting sequencing permits scientists to skip the step of synthesizing a sequence-specific primer to select desired strands. Instead, the scientist would specify the sequence of interest to a computer, which would control the nanopore device, which would be used in the strand selection process to physically separate desired strands from a sample.

### B. Substrate-Based Sequencing

In some embodiments, provided herein is a system for substrate-based sequencing and subsequent isolation of desired nucleic acids. In some embodiments, provided herein is a method for isolation of desired nucleic acids that depends, in part, on substrate-based sequencing. As used herein, the term “substrate-based sequencing” refers to any sequencing technology in which the nucleic acids to be sequenced are localized, directly or indirectly, to a specific spatial position on a substrate. In some embodiments, substrate-based-sequencing is used to determine the sequence of an individual nucleic acid strand, which is localized at a specific spatial location on a substrate. For example, in some embodiments the nucleic acids to be sequenced are distributed spatially within channels, such as microchannels or nanochannels. As another example, in some embodiments the nucleic acids to be sequenced are tethered to specific locations on a solid substrate.

In some embodiments, the nucleic acids are amplified, such as by PCR or isothermal amplification techniques, and subjected to synthesis reactions in which labeled nucleotides or chemical reactions based upon the incorporation of a particular nucleotide can be imaged or otherwise detected (e.g., by pH changes, detection of reaction byproducts, etc.) to determine the sequence of the nucleic acid strand. The nucleic acids are amplified, such as by PCR (e.g. bridge amplification) or by isothermal amplification methods, and subjected to synthesis reactions in which labeled nucleotides or chemical reactions based upon the incorporation of a particular nucleotide can be imaged or otherwise detected to determine the sequence of the nucleic acid strand. Substrate-based sequencing methods include, for example, sequencing-by-synthesis methods. Sequencing-by-synthesis methods generally use a solid support containing microchannels or wells in which the sequencing reaction occurs. In general, sequencing-by-synthesis methods rely on high sequence coverage (e.g. massively parallel sequencing) of millions to billions of short nucleotide sequence reads (e.g. 50-300) nucleotides).

In some embodiments, provided herein is a system for substrate-based sequencing and subsequent isolation of desired nucleic acids. In some embodiments, provided herein is a method for isolation of desired of nucleic acids that may be performed using a system as described herein. The methods for isolation of desired nucleic acids comprise performing a substate-based sequencing method, followed by selectively releasing desired nucleic acid strands from the substrate. In some embodiments, the desired nucleic acid strands are selectively released from the substrate. In some embodiments, the desired nucleic acid strands are accurate nucleic acid strands. Accordingly, in some embodiments the methods comprise selectively releasing accurate nucleic acid strands from the substrate, thereby leaving inaccurate nucleic acids bound to the substrate. In some embodiments, the desired nucleic acid strands are inaccurate nucleic acid strands. Accordingly, in some embodiments the methods comprise selectively releasing inaccurate nucleic acid strands from the substrate, thereby leaving accurate nucleic acids bound to the substrate.

In some embodiments, the system comprises substrate-based sequencing device. In some embodiments, the device comprises a substrate. The surface of the substrate may comprise any suitable material. In some embodiments, the surface of the substrate is porous. In some embodiments, the surface of the substrate is non-porous. In some embodiments, the surface comprises a material selected from glass, silicon, poly-L-lysine coated materials, nitrocellulose, polystyrene, polyacrylamide, cyclic olefin copolymers (COCs), cyclic olefin polymers (COPs), polypropylene, polyethylene and polycarbonate. In some embodiments, the surface comprises glass.

In some embodiments, the nucleic acids are bound to the surface of the substrate. In some embodiments, the substrate surface comprises an array of cleavable anchors. The term “cleavable anchor” refers to any suitable moiety bound to the surface of the substrate (e.g. through covalent or non-covalent interactions) that serves as attachment sites for nucleic acids to be sequenced (e.g. template nucleic acids). In some embodiments, the cleavable anchors comprise nucleic acids. For example, nucleic acids added to the substrate and/or nucleic acids amplified on the substrate (e.g. during bridge amplification) may bind to the cleavable anchors (e.g. by hybridization). In some embodiments, the cleavable anchors comprise beads. In some embodiments, the beads are immobilized (e.g. covalently bound) to the surface of the substrate. In some embodiments, the beads are not immobilized. In some embodiments, the spatial location and type of cleavable anchor at each spatially defined location within the substrate is known, such that the type of cleavable anchor can be affiliated with a given sequence of nucleic acid bound to the anchor. Accordingly, following sequencing of the nucleic acids on the substrate, specific (e.g. accurate) nucleic acids are released from the substrate by application of an appropriate stimulus to induce cleavage of the desired subpopulation of cleavable anchors affiliated with the accurate strands.

In some embodiments, the system for substrate-based sequencing comprises a mechanism for applying the stimulus to the desired subpopulation of cleavable anchors, or to the desired subpopulation of nucleic acids themselves to induce release of the nucleic acids from the substrate. For example, the system may comprise an light source (e.g. ultraviolet light source). In some embodiments, the light source (e.g. ultraviolet light source) controls light in a targeted manner to selectively cleave the desired cleavable anchor(s) from the surface of the substrate. For example, the light source may deliver light in a targeted manner, including adjusting factors including light intensity, light wavelength(s), the number of photons, the spatial location of the substrate, the size of the light beam, the duration for which the light is delivered, whether the light beam is a propagating mode or evanescent, whether the light source delivers a single photon excitation, a two-photon excitation, a three-photon excitation, or a multi-photon excitation including multi-photon excitation from photons of distinct wavelength, whether the light source is incoherent, pulsed or not pulsed, coherent, ultrafast, or a combination of the above characteristics. In some embodiments, the light source (e.g. ultraviolet light source) delivers light in a targeted manner, such as delivering a desired wavelength, a desired number of photons, or a desired target energy level to the substrate. As additional examples, the light source may deliver the light to a targeted spot on the substrate (e.g. a specific spatial location), deliver a specific size of light beam to the substrate (e.g. generate a light spot of a specific size) on the substrate. Variation of such factors may result in targeted release of a given subset of cleavable anchors from the substrate. For example delivery of a first targeted stimulus (e.g. a first wavelength, a first energy level, a first location on a substrate, etc.) results in cleavage of a first subpopulation of cleavable anchors or a first subpopulation of desired nucleic acid strands. Delivery of a second targeted stimulus (e.g. second wavelength, second energy level, delivery to a second location on the substrate, etc.) results in cleavage of a second subpopulation of cleavable anchors or a second subpopulation of desired nucleic acid strands.

In some embodiments, the light source may be capable of generating light of a variety of wavelengths. For example, in some embodiments one population of cleavable anchors is cleaved by a first wavelength of light, whereas a second population of cleavable anchors is cleaved by a second wavelength of light. Accordingly, in some embodiments the system comprises a light source that applies ultraviolet light of the desired wavelength to the desired strands on the substrate. In some embodiments, the system is capable of applying ultraviolet light of various wavelengths, wherein different wavelengths are used to release strands containing different cleavable anchors. In some embodiments, the system further comprises a UV filter.

In some embodiments, the cleavable anchors may be light sensitive. “Light” here refers to electromagnetic radiation in the far infrared, infrared, near infrared, visible, ultraviolet, or extreme ultraviolet spectrum ranging from a wavelength of 100 microns to a wavelength of 10 nanometers. Light-sensitive anchors are also referred to herein as “photocleavable”. “photocleavable linkers”, or “photolinkers”. The term “photocleavable” refers to an anchor that can be cleaved from the surface of the substrate by application of light of a certain wavelength, for example, ultraviolet (UV) light. Accordingly, application of light will cleave the anchor from the substrate, thereby releasing the desired nucleic acids (e.g. the nucleic acids bound to the anchor). In some embodiments, multiple ranges of light may be applied to sequentially cleave specific subpopulations of anchors. Following each sequential stimulus (e.g. each application of light), the desired subpopulation of nucleic acids can be collected prior to applying the next stimulus. For example, following sequencing a first subset of desired nucleic acid strands can be released from the substrate by using a targeted illumination machine to apply the appropriate stimulus (e.g. the appropriate wavelength of light) to the desired subset of anchors attached to the nucleic acid strands to be isolated. The first subset of nucleic acid strands are thus released and can be collected. Following isolation of the first subset, a second subset may be isolated (e.g. by applying a second stimulus, such as a second appropriate wavelength of light) to release the second subset of desired nucleic acids. Third subsets, fourth subsets, etc. can be isolated and collected in a similar manner. In some embodiments, an amplification step (e.g. PCR, isothermal amplification, etc.) may be performed to further enrich the number of desired nucleic acid strands following isolation.

In some embodiments, the photocleavable linker (i.e. “photolinker”) is any linker that is sensitive to light, including UV light, single-photon exposure, or multi-photon exposure. In some embodiments, the photolinker is cleaved using single-photon exposure. In some embodiments, the photolinker is cleaved using multi-photon exposure. In some embodiments, the multi-photon exposure comprises two-photon exposure. In some embodiments, the multi-photon exposure three-photon exposure. Any suitable wavelength(s) may be selected to cleave the photolinker. For example, for two-photon excitation the laser wavelength may be approximately 650 nm to 800 nm. As another example, for three-photon excitation the laser wavelength may be approximately 960 nm to 1050 nm. In some embodiments, multi-photon exposure is achieved by using an ultrafast laser, such as a femtosecond laser. In some embodiments, multi-photon exposure is achieved by the presence of an upconverting material, such as upconverting nanoparticles, which are flowed into the substrate during the cleaving step. In some embodiments, the upconverting nanoparticles are organic or inorganic. In some embodiments where multi-photon exposure is used, the photolinker is selected based upon its ability to absorb multi-photon stimuli. For example, a suitable photolinker for use with multi-photon exposure-based cleavage is 7-diethylaminocoumarin. Suitable photolinkers for use in the methods described herein, including those sensitive to single photon absorption or multi-photon absorption (two-photon absorption, three-photon absorption) are described in Klan et al., Chemical Reviews (2013) 113, 119-191, the entire contents of which are incorporated herein by reference.

In some embodiments, more than one photolinker is used. For example, in some embodiments a given strand comprises multiple photolinkers (e.g. two photolinkers, three photolinkers), thus increasing the probability of a cleavage event per incident excitation event occurring. In some embodiments, a nucleic acid strand is cleaved from the substrate without requiring a photolinker. For example, in some embodiments a stimulus can be applied to directly cleave the strand itself. For example, in some embodiments a stimulus is applied which breaks covalent bonds within the strand itself, thereby releasing at least a portion of the strand from the substrate.

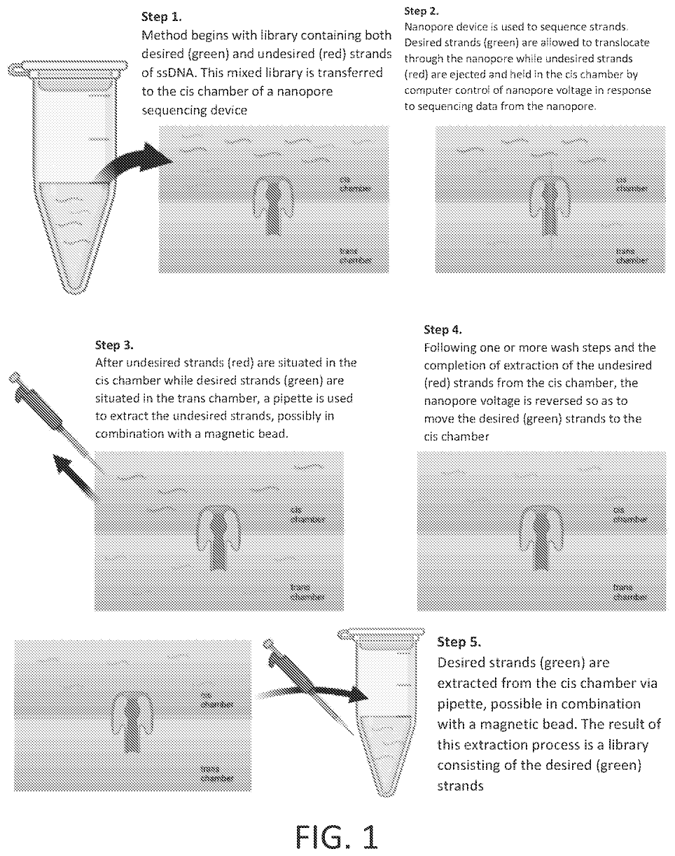

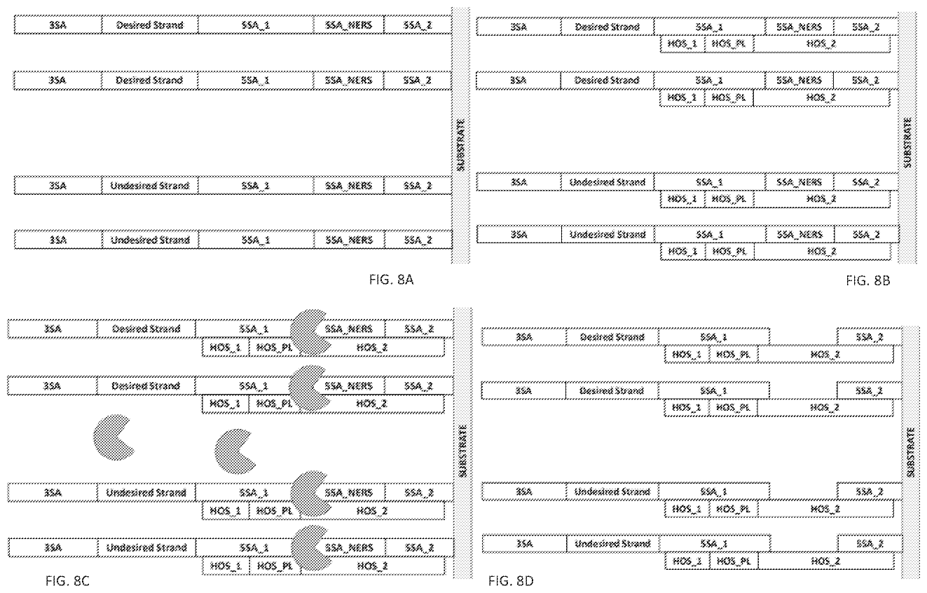

In some embodiments, the strand contains a sacrificial segment which remains attached to the substrate, whereas the remainder of the strand is released. For example, in some embodiments a sacrificial segment of about 20-100 bases (e.g. about 20, about 25, about 30, about 35, about 40, about 45, about 50, about 55, about 60, about 65, about 70, about 75, about 80, about 85, about 90), about 95, or about 100 bases) may remain attached to the substrate following cleavage. In such embodiments, the portion of the strand that is released from the substrate is still considered to be an “accurate” nucleic acid strand. In some embodiments, multi-photon excitation (e.g. two-photon or three-photon excitation) is used to break covalent bonds within the nucleic acid strand. In some embodiments, the strands also contain a cleavage site, such as for a restriction enzyme or a nickase, for subsequent clean-up after the desired strands are cleaved and removed (e.g. washed) from the substrate. In some embodiments, the excitation (whether single-photon or multi-photon. e.g. two-photon or three-photon) is delivered via total internal reflection, which reduces the excitation volume to an evanescent wave near the surface of the substrate, thereby further limiting the excitation volume. In some embodiments, nucleic acid strands on a sequencing substrate can be converted into photocleavable strands that can be targeted for isolation by directed UV light. FIG. 8 is a schematic of one such strategy. The schematic demonstrates two mechanisms that can be used. 1) photocleavage, which can be selectively directed at desired nucleic acid strands with optics and 2) enzymatic cleavage, which is applied uniformly to all nucleic acid strands. In the schematic in FIG. 1, the 5′-sequencing adapters attached to the flow cell and to all nucleic acid strands contain a recognition sequence for a nicking enzyme. After sequencing and removal of the extended sequencing primer, the nucleic acid strands attached to the flow cell are single-stranded as in FIG. 8A. An oligonucleotide that contains a photocleavable linker and is complementary to the 3′-end of the 5′-sequencing adapter is hybridized to the nucleic acid strands on the flow cell (FIG. 8B). Next, a nicking enzyme is introduced to break a phosphodiester bond between two bases downstream of the nicking enzyme recognition sequence found in the 5′-sequencing adapters attached to all strands (FIG. 8C). At this point, none of the strands dissociate from the flow cell, because the 3′- and 5′-ends of the 5′-sequencing adapters are held together by the hybridized photocleavable oligonucleotide (FIG. 8D). Finally, the desired strands are selectively exposed to UV light, photocleaving the oligonucleotides that are hybridized to the desired strands (FIG. 8E). This results in selective dissociation of the desired strands from the surface of the flow cell, because the region of the 5′-sequencing adapter that is contiguously hybridized to the photocleavable oligonucleotide is no longer sufficiently long to prevent spontaneous melting (FIG. 8F). Thus, the flow cell can be washed to recover the desired strands, while the undesired strands remain attached to the flow cell surface (FIG. 8G). A nicking enzyme is just one example of a cleavage mechanism that could be used for the application described above. Alternatives include restriction enzymes. CRISPR systems (e.g. Cas9 used in combination with a guide RNA that targets the 5′-sequencing adapter), transposases, programmable integrases, and targeted cleavage of a uracil base in the 5′-sequencing adapter with uracil DNA glycosylase and an appropriate endonuclease (e.g. endonuclease VIII).

In some embodiments, the stimulus to release the desired anchors from the substrate is heat. For example, in some embodiments spatially localized heating is used to cleave a meltable linker which is used to bind the nucleic acid strands to the substrate. The meltable linker may be a denaturable protein-ligand complex such as biotin-streptavidin. Alternatively, there are chemical cross-linkers that are known to be heat-sensitive and reversible such as formaldehyde-based cross-linkers. In some embodiments, spatially localized application of infrared light is used to achieve spatially localized heating, or to more directly cleave hydrogen bonds formed between nucleic acids by infrared light chosen in consideration of wavelengths well suited to nucleic acid hydrogen bond absorbance peaks. In some embodiments, spatially localized heating is used for the purpose of either cleaving the linker to the substrate or for de-hybridization of desired nucleic acid strands hybridized to the sequencing substrate. Spatially localized heating may be achieved by microheater arrays fabricated into the substrate or placed in contact with the substrate. Spatially localized heating may also be achieved by application of spatially localized infrared electromagnetic radiation.

In some embodiments, spatially localized heating is used to melt nucleic acid strands which are hybridized to an nucleic acid strand which is immobilized to a sequencing substrate, for the purpose of isolating the hybridized strand. Suitable platforms for isolation by melting include sequencing-by-synthesis reactions which produce a complementary hybridized strand, such as the bridge PCR technology utilized in the Illumina/Solexa product line (e.g. MiSeq, HiSeq, NovaSeq), in single molecule sequencing technology such as that of Pacific Biosciences SMRT sequencing. Paciific Biosciences HiFi sequencing. SeqLL/Helicos, or in sequencing-by-binding technology such as that of Omniome (now Pacific Biosciences Onso). Additional suitable platforms include those commercialized by GeneMind Biosciences, which are highly similar to SeqLL/Helicos technology.

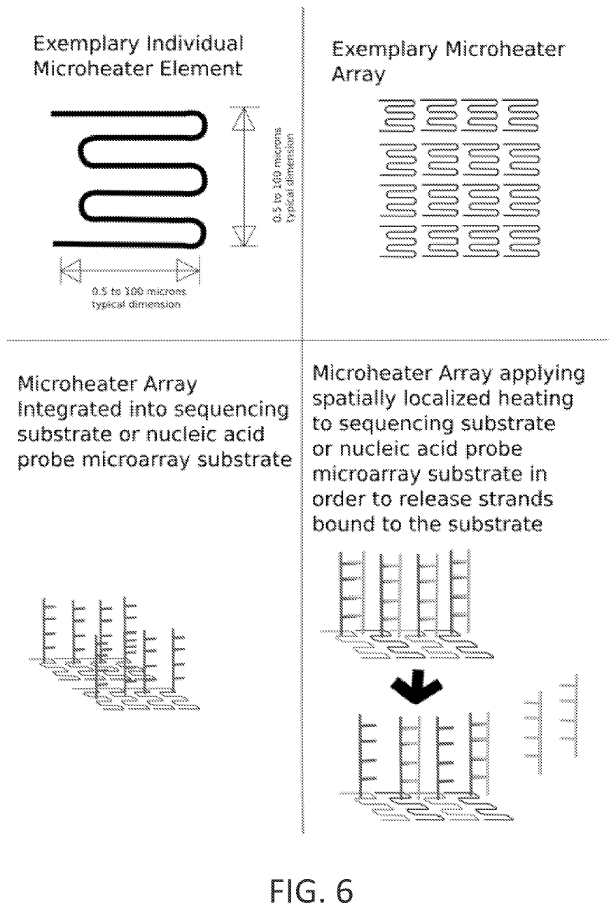

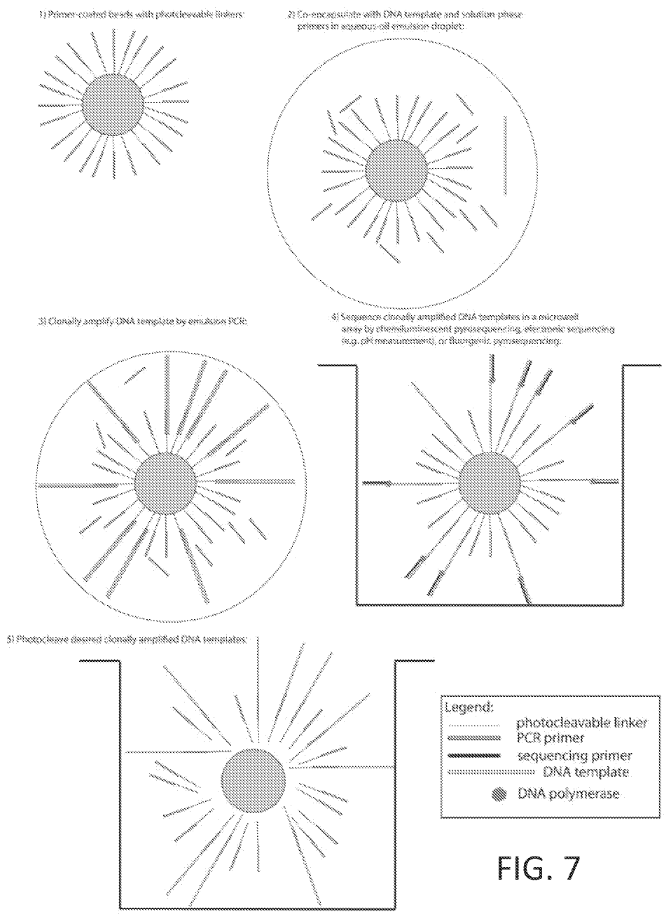

Spatially localized light may be applied to the substrate via a variety of techniques. In some embodiments, what we will refer to herein as a “light source array” such as ultraviolet micro-light emitting diodes may be used to perform photocleavage in a spatially controlled manner (Wu. Meng-Chyi, and I-Ting Chen. “High-Resolution 960×540 and 1920×1080 UV Micro Light-Emitting Diode Displays with the Application of Maskless Photolithography.” Advanced Photonics Research 2.7 (2021): 2100064, including FIG. 6). The light source array, such as micro-light emitting diodes, may also emit infrared or visible wavelength light. Alternatively, the light source array may be formed by an organic light emitting diode array, a vertical cavity surface-emitting laser array (Harasaka. Kazuhiro, et al. “Low thermal resistance 780 nm GaInPAs/GaInP 40ch VCSEL array for laser printers.” 17th Microopies Conference (MOC). IEEE. 2011), a quantum dot display, a liquid crystal display with a backlight (including an ultraviolet backlight), other spatial light modulator with backlight (including an ultraviolet backlight), or a digital micromirror array with backlight (including an ultraviolet backlight). The light source array may consist of a single light source, a million light sources, fifty million light sources, or many more than fifty million light sources, and may be as small as 50 microns on the diagonal. 1 millimeter on the diagonal. 100 millimeters on a diagonal, up to and including the size of an entire semiconductor wafer (upwards of 675 millimeters or more on the diagonal). The light source array may be brought into close contact with the sequencing substrate, such as a separation of zero microns, one micron, five microns, ten microns, fifty microns, one hundred microns, five hundred microns, up to and beyond one millimeter away, so as to project the pattern of light directly onto the sequencing substrate without need for a separate optical system (such as an objective lens). The light source array may also be focused onto the sequencing substrate by an optical system featuring a focal plane at the light source array and a focal plane at the sequencing substrate. A computer may provide a control signal to electronics which control the emission of light from each light source within the light source array and thereby determine which light sources should be on or off and what the intensity of each light source should be. The computer may also change the control signal of which light sources should be on or off and what their intensity should be so as to accommodate misalignments between the sequencing substrate and the light source array without need for mechanical movement of the substrate with respect to the light source array. Distinct choices of light intensity may be used to control the relative number of strands liberated from the substrate within each spatially localized region, if multiple strands are present within each spatially localized region. Microlenses, zone plate lens arrays, and micromirrors may be utilized to assist in directing the light to the desired spatial locations, such as in reducing the numeric aperture of emission from the light source so as to promote collimated emission and to promote spatial localization of the emitted light onto the substrate of interest. A gasket array situated on top of the light emitting diode array, such as an array of holes drilled into a thin substrate, such as a one micron, ten micron, fifty micron, or five hundred micron thick silicone gasket may be used to assist in controlling stray light to prevent undesired exposure of neighboring spatial regions or other undesired. The material(s) used for fabricating the gasket will typically be compliant so as to accommodate variations in the surface roughness of both the sequencing substrate and the light source array, and the material(s) used for the gasket may be designed to include materials which are absorptive or which can downconvert (or upconvert) the wavelengths of light used for photocleavage or de-hybridization into wavelengths which are not relevant for the purpose of performing photocleavage or de-hybridization. If there is a mismatch between the pitch of the light sources and the pitch of the desired spatially localized regions on the sequencing substrate or if the sequencing substrate is larger than the light source array, the sequencing substrate may be moved mechanically with respect to the light source array by a stage so as to accommodate repeated exposures of different regions the substrate to light from the light source array. In some embodiments, the microLED or organic LED array may be fabricated as part of the sequencing substrate itself.

In some embodiments, one or more wash steps may be performed prior to applying the source to liberate the desired strands from the substrate. Such wash steps may be employed prior to application of the targeted stimulus (e.g. ultraviolet light), or in between multiple applications of the stimulus (e.g. in between a first targeted stimulus that releases a first population of strands and a second targeted stimulus that releases a second population of strands).

In one embodiment, a single molecule real-time substrate-based-sequencing technology may be used. In some embodiments of single molecule real-time substrate-based-sequencing, a highly processive polymerase (e.g. DNA polymerase or RNA polymerase) is immobilized on a surface. Addition of the sample containing the template nucleic acids (e.g. the nucleic acids to be sequenced) results in binding of the polymerase to the template nucleic acid (e.g. a DNA template or an RNA template). The polymerase incorporates nucleotides modified with fluorescent labels to the template, and this processes is monitored in real-time with a fluorescence detection system to sequence the template. In some embodiments, the polymerase is immobilized to the substrate using conjugation chemistry. For example, polymerase molecules can be immobilized to the surface of a substrate biotin-streptavidin bioconjugation chemistry. In some embodiments, biotinylated reagents that include photocleavable chemical linkers may be used, which allow release of biotinylated proteins from surfaces upon exposure to ultraviolet (UV) light.

In some embodiments, the template nucleic acid may be prepared such that the template comprises a biotinylated polymerase conjugated to one end of the template sequence. The substrate may comprise a biotinylated surface, to which streptavidin may be bound. The template is thus anchored to the surface through interactions between the biotinylated DNA polymerase and the streptavidin bound to the substrate surface. Following sequencing, the desired templates (e.g. templates having an accurate strand) may be released from the substrate by photocleavage. As described above, the spatial location of the desired strand is known, such that the appropriate light or heat may be applied only to the desired spatial locations on the substrate to induce cleavage of desired strands, while undesired strands remain bound to the substrate. Thus, targeted release of individual polymerase-bound templates is achieved by high-resolution direction of light or heat after sequencing to identify the desirable template strands. The released material may be separated by suitable purification methods, including column- or bead-based purification. The polymerase may be deactivated, thus resulting in an isolated, desired nucleic acid strand. For example, the polymerase may be deactivated by heating. In embodiments wherein a polymerase or another suitable binding agent immobilized on the substrate is bound to a template nucleic acid, freeing the polymerase (or the suitable binding agent) to release the polymerase-bound nucleic acid strand is considered the equivalent of selectively releasing the nucleic acid strand itself. In other words, selectively isolating the target nucleic acid may comprise releasing the polymerase or other binding agent holding the desired template to the substrate, and may further comprise subsequently deactivating the binding agent (e.g. deactivating the polymerase) to result in an isolated, accurate nucleic acid strand.

In some embodiments, sequencing of small colonies of clonally amplified DNA templates, rather than single molecules, may be employed. Such methods are referred to herein as “clonal” substrate-based sequencing. In some embodiments of clonal substrate-based sequencing, individual template strands are captured on surface-immobilized oligonucleotide primers by hybridization and clonally amplified using surface-immobilized primers by solid-phase PCR (e.g. bridge PCR). A variety of sequencing chemistries can then be used to sequence the clonally amplified DNA templates. In some embodiments, reversible terminator chemistry methods may be used to sequence the clonally amplified DNA. In some embodiments, oligonucleotide primers may be immobilized on surfaces (e.g. on the surface of the substrate) using photocleavable chemical linkers that allow targeted release of the oligonucleotides from the surface by exposure to light. Thus, templates conjugated to the oligonucleotide primers immobilized on the surface of the substrate may also be isolated by exposure to light, and subsequently separated from the primer. Thus, similar to the system described above for use in single-molecule isolation, an optical system that allows targeted release of individual clonal amplicons by high-resolution direction of light after sequencing an array of clones to identify clonal DNA template clones with the desired sequence may be used. The released material would include both primers and the desired primer-conjugated template, which can be readily separated (e.g. by size selection).

Substrate-based-sequencing and subsequent isolation of desired nucleic acid strands can be performed using a computerized process. For example, the computer may direct any one or more steps in the process of sequencing and isolating desired nucleic acids from the substrate. For example, the computer may direct the sequencing method (e.g. sequencing-by-synthesis method), determine the sequence of the template nucleic acid strand, and/or control the application of the stimulus (e.g. ultraviolet light) to the desired area on the substrate to induce release of the accurate nucleic acid strands. Accordingly, in some embodiments the system for substrate-based sequencing further comprises a computer. In some embodiments, the system for substrate-based sequencing comprises a substrate-based sequencing device, as described above, and a computer. The computer may comprise a memory and a processor, wherein the memory encodes instructions that dictate that the processor perform a given task. In some embodiments, the computer employs and algorithm to determine the sequence of nucleic acid strands. The algorithm may additionally compare the sequence of a given nucleic acid strand to the intended sequence to determine whether a given nucleic acid strand is desirable. For example, the algorithm may determine whether a given sequence has a desired sequence identity, the desired length, a desired methylation status, etc. The algorithm may determine that a sequence has any combination(s) of desired properties with any likelihood, including combinations which make use of conditional relationships, logical relationships, control flow, or state, or comparison to other strands, or information stored in local or remote databases. The algorithm may be encoded in software, which may be stored in a memory of the computer. Alternatively, the algorithm may be encoded in hardware, which may be operably connected to the computer prior to use (e.g. inserted as a CD-ROM, external disc, external hard drive, etc.). Upon determination of whether the strand is desired (e.g. accurate), the computer may instruct the process to apply the appropriate stimulus (e.g. the appropriate wavelength, intensity, and location of light or location and temperature of heat) to the cleavable anchor bound to the strand, thereby releasing the strand from the substrate surface. The computerized process may be fully autonomous, or the computerized process may pause and ask for decisions from a human operator during one or more steps.

In some embodiments, the system comprises software. In some embodiments, the software is stored on a computer. For example, the software may be stored in a memory of the computer. In some embodiments, the software may be stored on an external medium, such as a CD-ROM, a floppy disk, a hard drive, a DVD, a Blu-ray disk, etc., which may be suitably connected to the computer prior to executing the software stored therein. In some embodiments, the software is designed to execute one or more tasks in a method of nanopore sequencing as described herein. In some embodiments, the software instructs a processor to execute a given task. In some embodiments, the software stores machine readable instructions. For example, in some embodiments the software stores machine readable instructions that instruct the processor to execute a given task. The machine-readable instructions may be one or more executable programs or portion(s) of an executable program for execution by a processor.

In some embodiments, the software collects and analyzes data from the substrate-based sequencing device. For example, in some embodiments the software collects and analyzes data regarding the sequences, lengths, or other characteristics of nucleic acid strands at a given spatial location on the substrate. In some embodiments, the software analyzes the sequence data, such as by comparing the sequence of a given nucleic acid strand to the sequence of a desired (e.g. accurate) nucleic acid strand. In some embodiments, the software actuates other components of the system to control the isolation of desired strands from undesired strands. For example, the software may instruct the processor to perform one or more functions, thereby controlling isolation of desired strands from undesired strands. For example, the software may instruct the processor to apply an ultraviolet light stimulus to one or more spatial locations on the substrate, thereby releasing the desired (e.g. accurate) strand(s) from the substrate.

In some embodiments, nucleic acids may be segregated into separate fluid volumes prior to isolation of the desired nucleic acid. The term “separate fluid volumes” indicates that preferential extraction of the content of one fluid volume compared to a second fluid volume can occur. Separate fluid volumes need not be physically disparate fluid volumes. For example, separate fluid volumes may be shared within the same solution, and yet preferential extraction from one fluid volume is still possible. In some embodiments, nucleic acids may be segregated into separate fluid volumes based upon features such as charge, size, structure (e.g. secondary structure, tertiary structure, etc.), or other suitable features or combinations thereof. For example, in some embodiments electrophoresis may be performed to drive nucleic acids having a desired charge towards one end of a fluid, thus generating a separate fluid volume “A” from which preferential extraction of the desired nucleic acids can occur.

The process of extracting desired nucleic acids need not be conducted perfectly reliably. The separation process may merely be enrichment, such as an extraction of the contents of one fluid volume where we have an increased likelihood of extracting from fluid volume “A” compared to fluid volume “B”. For example, the extraction may have at least a 51% likelihood of extracting from fluid volume “A” and a 49% likelihood or less of extracting from fluid volume “B”. For example, an extraction may have at least a 51%, at least a 60%, at least a 70%, at least an 80%, at least a 90%, at least a 95%, or a 99% or higher likelihood of extracting from fluid volume “A”.

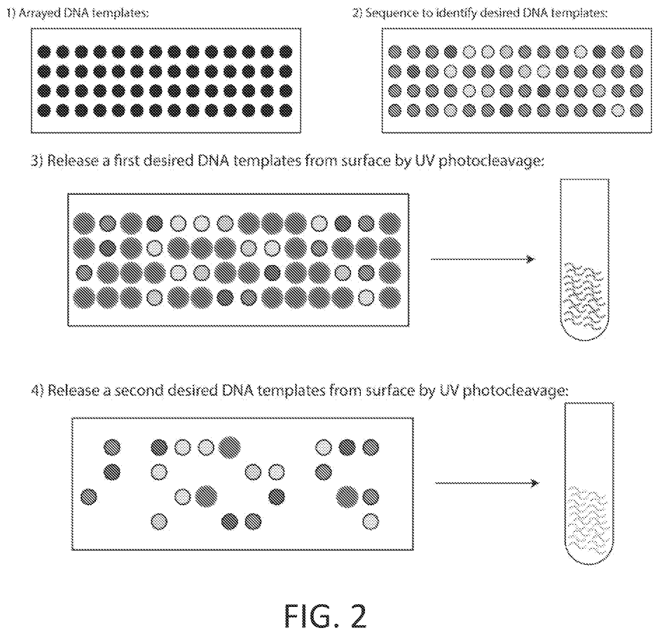

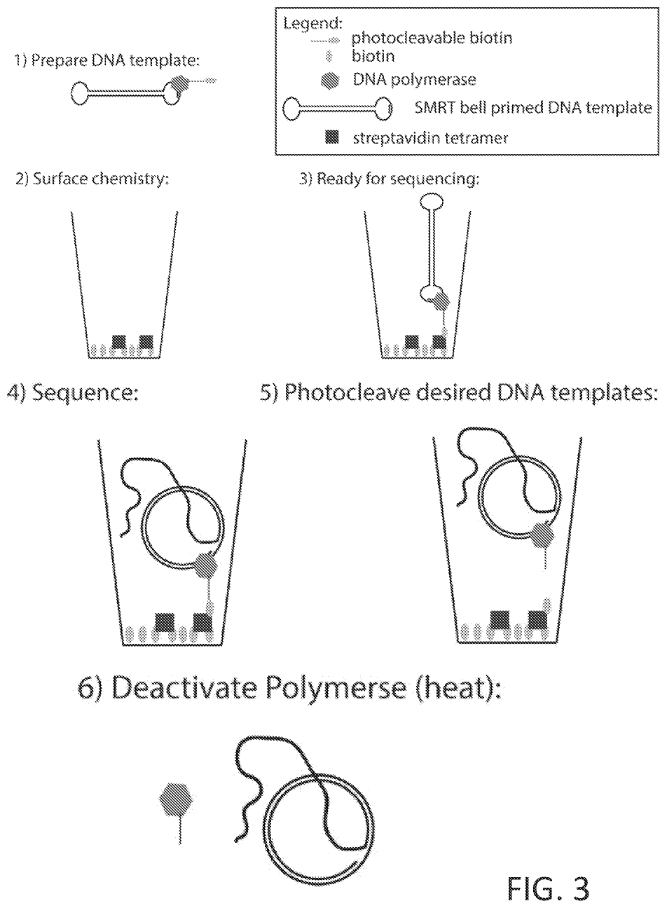

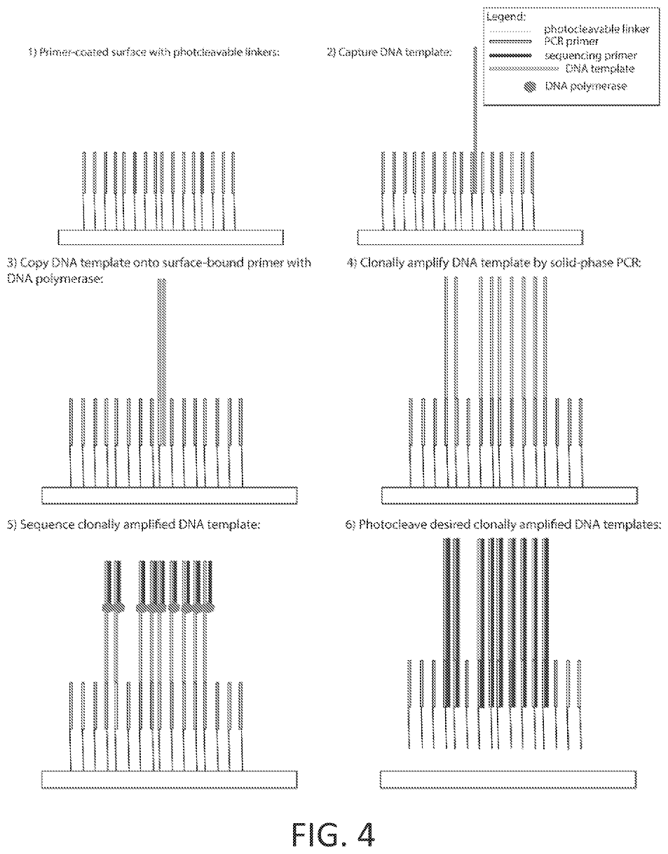

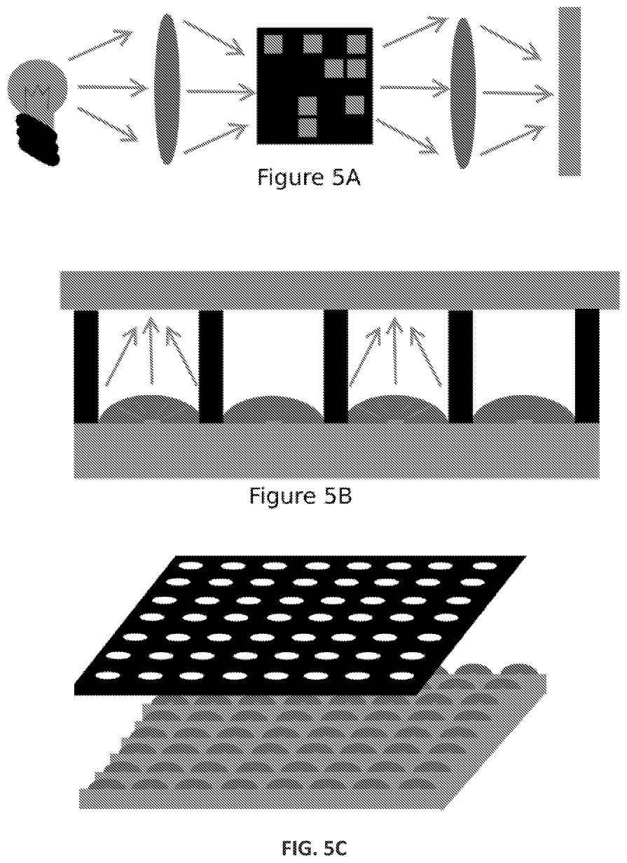

A variety of suitable sequencing methods and technologies may be used to determine the sequence of the nucleic acid strands. For example, the sequencing method may be a next generation sequencing technology. The term next generation sequencing, or “NGS”, refers to a variety of sequencing techniques that permit simultaneous sequencing of millions of nucleic acid sequences, and is otherwise referred to as high-through put sequencing or massively parallel sequencing. Suitable NGS technologies are reviewed in, for example. Zhong et al., Ann Lab Med. 2021 Jan.; 41 (1): 25-43, and Slatko et al., Curr Protoc Mol Biol. 2018 Apr.; 122 (1): e59, the entire contents of each of which are incorporated herein by reference. Suitable NGS technologies include, for example, second generation sequencing technologies such as pyrosequencing (e.g. 454 pyrosequencing), ion torrent sequencing (e.g. including various platforms sold by Thermo-Fischer, including the Ion Torrent System. Ion Personal Genome Machine™. Ion Proton™ Ion S5, and Ion S), and bridge PCR-based amplification methods. Additional pyrosequencing methods include technologies marketed by Genapsys, including Genapsys GS111. In general, pyrosequencing methods captures pyrophosphate (PPi) release and uses it as an indicator of specific base incorporation. Ion torrent sequencing methods rely on hydrogen ion detection technology, which detects the release of protons during incorporation of nucleotides into the nucleic acid strand during synthesis. Suitable bridge PCR-based amplification technologies include various Illumina platforms, such as MiSeq, MiniSeq, MiSeq, HiSeq, and NextSeq platforms. For example, an Illumina sequencing platform based on sequencing-by-synthesis may be used in a method comprising generating sequences of DNA templates, and releasing desired strands using UV-photocleavage as shown in FIG. 2 or as shown in FIG. 4. Additional suitable NGS technologies include third generation sequencing technologies, which have been developed to overcome challenges with second generation technologies including short sequence reads leading to sequence gaps, alignment issues, and/or PCR artifacts. Suitable third generation NGS technologies include, for example, technologies developed by Pacific Biosciences (e.g. PacBio) single molecule real-time (SMRT) technology. PacBio SMRT technology does not require amplification. Rather, adapters used in library preparation have a hairpin structure to ensure that the double-stranded DNA fragments become circular after ligation to form the SMRTbell template (see, for example. FIG. 3). The bases are sequenced by synthesis in real time on a chip containing millions of zero mode waveguides (ZMWs), which are nanowells several nanometers in diameter and approximately 100 nm in depth. The template molecule and DNA polymerase are immobilized at the bottom of each ZMW. During the sequencing reactions, the complementary strand of the template is elongated by DNA polymerase with fluorescently labeled deoxyribonucleotide triphosphates. camera sensor inside of the machine, such as a focal plane array sensor, such as a camera CCD or CMOS imaging sensor, EMCCD imaging sensor, or other imaging sensor, captures and records the fluorescent signals in real-time. Suitable platforms marketed by PacBio include the RS, RSII, Sequel, Sequel II, and any subsequent or prior systems based on the SMRT technology utilizing zero-mode waveguides or zero-mode waveguide based optical nanopores.

Additional suitable platforms other than those described above may be used in accordance with the methods described herein. For example, other additional substrate-based-sequencing technologies and platforms include electronic DNA sequencing technology marketed by Roswell Biotech e.g. US10913966. Such technology may utilize more than one photocleavable or meltable linker to isolate the desired nucleic acid strands. In some embodiments, additional suitable SBS technologies include DNA nanoball sequencing technology. DNA nanoball sequencing technology is a high throughput sequencing technique that relies on rolling circle amplification to amplify small fragments of genomic DNA into DNA nanoballs. Fluorescent nucleotides bind to complementary nucleotides and are then polymerized to anchor sequences bound to known sequences on the DNA template. Another example of a suitable SBS technology is the single molecule nucleic acid sequencing technology of SeqLL (formerly Helicos). e.g. U.S. Pat. No. 8,367,377B2. Another example of a suitable SBS technology is the DNA nanoball-based technology marketed by the Beijing Genomics Institute including BGISEQ-500, and MGISEQ-2000, formerly by Complete Genomics. e.g. US20190010542A1. An additional suitable platform is the sequencing-by-binding technology of Omniome (now Pacific Biosciences) e.g. U.S. Pat. No. 10,246,744B2. An additional suitable platform is the sequencing-by-hybridization technology of Nanostring named “Hyb-and-Seq”. e.g. EP3221469B1. An additional suitable platform is the multivalent binding composition for nucleic acid analysis by Element Biosciences, e.g. US20,220,186310A1.

Substrate-based nucleic acid assays which do not necessarily serve the purpose of determining the sequence of the nucleic acid strand directly but instead yield information regarding the identity of the target strands are also a suitable platform. Substrate-based nucleic assays operate by hybridizing target strands to probes which are linked to a substrate in a spatially-localized manner, as represented, for example, by the technology of ThermoFisher (formerly Affymetrix) GeneChip or Illumina Microarray/BeadArray. In other embodiments, substrate-based nucleic acid assays link the target strands to a substrate in a spatially-localized manner and then hybridize labeled nucleic acid probes to the strands, as is represented, for example, by the Nanostring nCounter system (e.g. US8415102B2). Probes used in substrate-based nucleic acid assays may provide information regarding the target strand sequence, target strand methylation status, which proteins the target strand binds to, or other information regarding the target strand.

If a non-sequencing substrate-based nucleic acid assay (“SBNAA”), such as those described above, is used, the hybridization status of the target and the probe may be determined and then the appropriate stimulus (e.g. heat or light) may be delivered to a desired area to cleave the desired regions on the array. Alternatively, it may be determined that there is a high certainty that the desired target strands are localized to a particular spatial location on the array and in this instance, it may not be necessary to observe the hybridization status of the target and probe. In some embodiments, cleaving the spatially-localized linkers or heating spatially-localized desired regions of the SBNAA (e.g. DNA microarray) may be performed without observing hybridization status. In some embodiments, the spatially-localized application of light or heat may either be computer-controlled, or it may be fixed in advance. In some embodiments, if spatially localized heating is used to de-hybridize the target strands from the probes and if microheaters are used to apply heat, then the microheaters may be fixed in advance of the experiment so as to not be chosen by a computer during the experiment. For example, microheaters may not have been fabricated in certain spatial locations on the substrate, or the microheaters may contain fuses which were earlier broken so as to prevent operation of the microheaters in specific regions. If light (e.g. infrared light or ultraviolet light based photocleaving) is utilized to recover the target strands, a physically fixed mask may be applied rather than a computer-controlled illumination source. The number of distinct probe sequences used in the SBNAA (e.g. microarray) may be small in number, such as one, or one dozen, or the number of distinct probe sequences may be large, such as ten thousand, or the number of distinct probe sequences may be very large, such as one million or one hundred million, or any number therein.

### EXAMPLES

### Example 1

### Photocleavage-by-Hybridization

This example provides data from an experiment using a photocleavage-by-hybridization approach. An Illumina TruSeq RNA-seq library was sequenced on an Illumina NovaSeq 6000 S4 flow cell. After sequencing, the flow cell was recovered and 100 mM sodium hydroxide was introduced to chemically melt any extended primers from the single-stranded DNA attached to the flow cell surface from two of the four lanes. The two flow cell lanes were then rinsed with Wash Buffer (20 mM Tris-HCl pH 7.9, 50 mM NaCl, 0.1% Tween-20). The RNA-seq library was subjected to paired-end sequencing, and the remaining nucleic acid strands were immobilized with the Illumina P5 flow cell adapter. The photocleavable oligonucleotide (PC-oligo) was then introduced to the two flow cell lanes in 2×SSC buffer (300 mM NaCl, 30 mM trisodium citrate pH 7) and incubated at room temperature for 30 minutes. The sequence of PC-oligo, purchased as a custom product from Integrated DNA Technologies, is given below. PC indicates a photocleavable spacer containing a photolabile nitrobenzene group that absorbs UV light (300-400 nm):

(SEQ ID NO: 1)
5′-GAAGAGCGTCG
(SEQ ID NO: 2)
-PC-TAGGGAAAGAGTGTAGATCTCG-3′

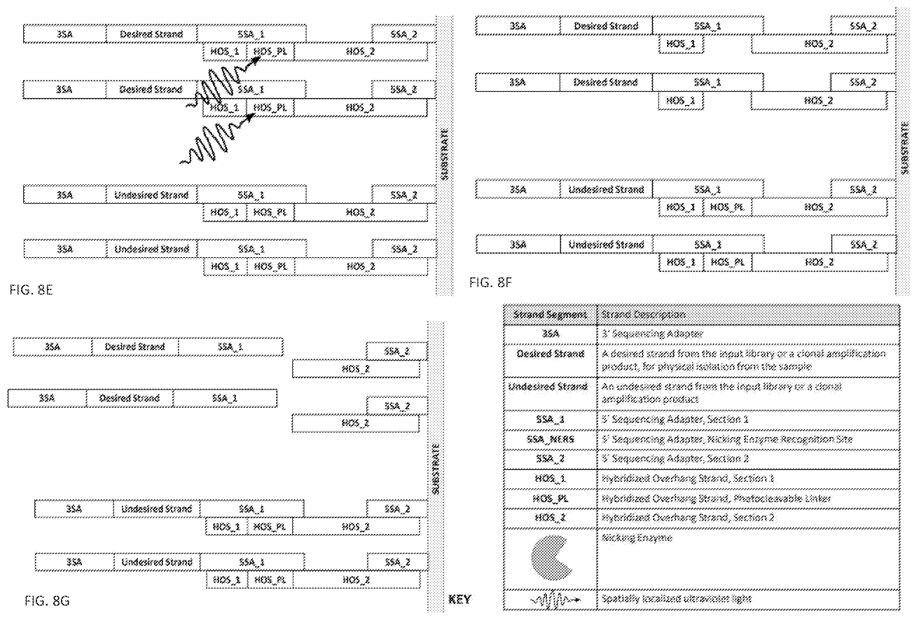

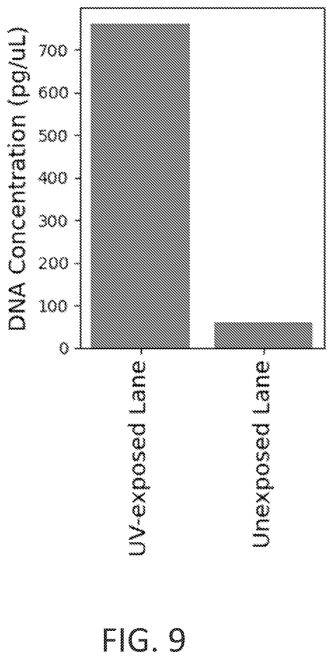

The PC-oligo is complementary to the P5 Illumina TruSeq adapter. Excess PC-oligo was washed from the two flow cell lanes with Wash Buffer. Next, 10 units of the nicking enzyme Nt. CviPII (New England Biolabs) was introduced to the two flow cell lanes in 1× rCutSmart Buffer (New England Biolabs) and incubated at 37 C for two hours. The enzymatic reaction mixture was washed from the flow cell with Wash Buffer. Next, one of the two flow cell lanes was exposed to UV light (UVP Blak-Ray B-100A UV lamp, 365 nm) for 10 minutes while the other lane was shielded from exposure with aluminum foil. After heating the flow cell for 10 minutes at 37C, liquid solution was recovered from both lanes of the flow cell. The recovered material from both lanes was then quantified by fluorometry using a Qubit ssDNA Assay Kit (ThermoFisher). The UV-exposed lane yielded substantially more DNA than the lane that was protected from UV exposure (FIG. 9).

### Example 2

A nanopore-based method of isolating nucleic acid strands from a mixed library is described in his example. A sample containing a mixed library (e.g. a library containing both accurate and inaccurate nucleic acid strands) can be applied to a first chamber of a nanopore sequencing device. The device can comprise a first chamber and a second chamber separated by a substantially impermeable membrane housing a plurality of nanopores. A flow of current can be induced through each nanopore, such that individual nucleic acid strands enter into the nanopores housed within the membrane. The nanopore sequencing device can comprise a plurality of electrodes, each electrode operably connected to a distinct nanopore within the substantially impermeable membrane. Accordingly, inducing a flow of current through each nanopore can comprise applying a voltage through each of the plurality of electrodes. The sequence of each individual nucleic acid strand can be determined as it passes through a nanopore, and each strand can be identified as accurate or inaccurate. For example, the nanopore sequencing device can comprise a plurality of sensors, each sensor recording a current passing through a single nanopore, such that the current passing through each nanopore is independently recorded. Determining the sequence of each individual nucleic acid strand as it passes through a nanopore can comprise recording the current passing through each nanopore (e.g. via the sensor), and determining the sequence of a given nucleic acid strand based upon the disruption of current that occurs as the nucleic acid strand passes through the nanopore. The desired nucleic acid strands can be isolated from the sample.

Isolating the desired nucleic acid strands from the sample can comprise modulating the voltage applied through each electrode operably connected to a nanopore containing an undesired nucleic acid strand, such that passage of the undesired nucleic acid strand through the nanopore is halted and/or reversed. The voltage applied through each electrode operably connected to nanopore containing a desired nucleic acid strand is not modulated, such that desired nucleic acid strands pass through the nanopores into the second chamber of the nanopore sequencing device. The desired nucleic acid strands can be isolated from the second chamber of the nanopore sequencing device. In some cases, the desired nucleic acids are not isolated from the second chamber, and instead the voltage applied to one or more electrodes can be reversed following removal of undesired strands from the first chamber, such that the desired nucleic acid strands housed within the second chamber are drawn through the nanopores into the first chamber. The desired nucleic acid strands can then be isolated from the first chamber.

In some cases, the voltage applied to each electrode operably connected to a nanopore containing an undesired nucleic acid strand can be reversed, such that the undesired nucleic acid strands are ejected into the first chamber of the nanopore sequencing device. The undesired nucleic acid strands can be removed from the first chamber.

### Example 3

A nanopore-based method of isolating nucleic acid strands from a mixed library is described in his example. A sample containing a mixed library (e.g. a library containing both accurate and inaccurate nucleic acid strands) can be applied to a first chamber of a nanopore sequencing device. The device can comprise a first chamber and a second chamber separated by a substantially impermeable membrane housing a plurality of nanopores. A flow of current can be induced through each nanopore, such that individual nucleic acid strands enter into the nanopores housed within the membrane. The nanopore sequencing device can comprise a plurality of electrodes, each electrode operably connected to a distinct nanopore within the substantially impermeable membrane. Accordingly, inducing a flow of current through each nanopore can comprise applying a voltage through each of the plurality of electrodes. The sequence of each individual nucleic acid strand can be determined as it passes through a nanopore, and each strand can be identified as accurate or inaccurate. For example, the nanopore sequencing device can comprise a plurality of sensors, each sensor recording a current passing through a single nanopore, such that the current passing through each nanopore is independently recorded. Determining the sequence of each individual nucleic acid strand as it passes through a nanopore can comprise recording the current passing through each nanopore (e.g. via the sensor), and determining the sequence of a given nucleic acid strand based upon the disruption of current that occurs as the nucleic acid strand passes through the nanopore. The desired nucleic acid strands can be isolated from the sample.

Isolating the desired nucleic acid strands from the sample can comprise applying a stimulus to selectively induce cleavage of desired nucleic acid strands from the nanopore. For example, nucleic acid strands can be connected to a linker, such as heat-sensitive or a light-sensitive linker (e.g. a photolinker). Strands can be permitted to pass through the nanopores until the linker is exposed. Desired nucleic acid strands can be released from the nanopore by selectively applying the stimulus (e.g. heat, or light) to the nanopores containing the desired nucleic acid strands, thereby cleaving the linker and releasing the desired strands. For example, the nucleic acid strands can be connected to a photolinker, and a light stimulus (UV light, one-photon, multi-photon) can be selectively applied to the desired strands, thereby cleaving the linkers and releasing the strands from the nanopore. The released accurate strands can then be isolated. In contrast, inaccurate strands are contained within the nanopore. Following selective release of desired strands, the voltage applied through each electrode operably connected to a nanopore containing an undesired nucleic acid strand can be reversed, such that the undesired nucleic acid strands are ejected into the first chamber of the nanopore sequencing device. The undesired nucleic acid strands can be removed from the first chamber.

### Example 4

A substrate-based method of isolating desired nucleic acid strands from a mixed library is described in this example. A sample comprising the mixed library can be provided to a substrate. The substrate can comprise a plurality of cleavable anchors at distinct locations on the surface of the substrate, such that individual nucleic acid strands bind to the cleavable linkers. A stimulus can be applied to the substrate to induce selective cleavage of the cleavable anchors bound to desired locations on the surface of the substrate, thereby releasing nucleic acid strands from the surface of the substrate. The released nucleic acid strands can be isolated, such as by washing. The sequence of the nucleic acid strands bound to the cleavable linkers can be identified prior to application of the stimulus, and each strand can be identified as accurate or inaccurate. The stimulus may be applied to induce selective cleavage of the cleavable anchors bound to desired nucleic acid strands, thereby selectively releasing the desired nucleic acid strands from the surface of the substrate. In contrast, the undesired strands are not cleaved, and therefore remain bound to the surface of the substrate.

The linkers can be photocleavable linkers (i.e. photolinkers). The stimulus applied to induce cleavage of the photolinkers can be light, including ultraviolet light, one-photon light, or multi-photon light (e.g. two-photon light, three photon-light). The wavelength of the light stimulus can be selected depending on the specific linker to achieve the desired cleavage of the linker. The light can be applied to specific spatial locations on the substrate, determined as containing an accurate nucleic acid strand (e.g. an accurate nucleic acid strand bound to the linker, which is bound at that location to the surface of the substrate).

The linkers can be heat-sensitive linkers, in which case heat is applied to specific spatial locations on the substrate to induce selective cleavage of accurate nucleic acid strands.

Following cleavage, the accurate nucleic acid strands can be isolated from the substrate and used for downstream methods.

### Example 5

A substrate-based method of isolating accurate nucleic acid strands from a mixed library is described in this example. A sample comprising the mixed library can be provided to a substrate. The substrate can comprise a plurality of nucleic acids bound to distinct locations on the surface of the substrate, without the use of cleavable linkers. The sequence of the nucleic acid strands bound to the substrate can be identified, and each strand can be identified as accurate or inaccurate. A stimulus can be applied to the substrate to induce selective cleavage of accurate nucleic acids from the surface of the substate. For example, multi-photon exposure can be applied to the substrate to selectively disrupt covalent bonds of desired nucleic acid strands, thereby releasing the desired nucleic acid strands from the surface of the substrate while leaving undesired strands bound to the substrate. In this method, a portion of desired nucleic acid strands, referred to as a “sacrificial segment”, remains on the surface of the substrate whereas the remainder of the desired nucleic acid strands (e.g. the portion released by disruption of the covalent bonds) is released. The desired nucleic acid strands may then be isolated from the substrate, such as by one of more wash steps, and used for downstream methods.

### Example 6

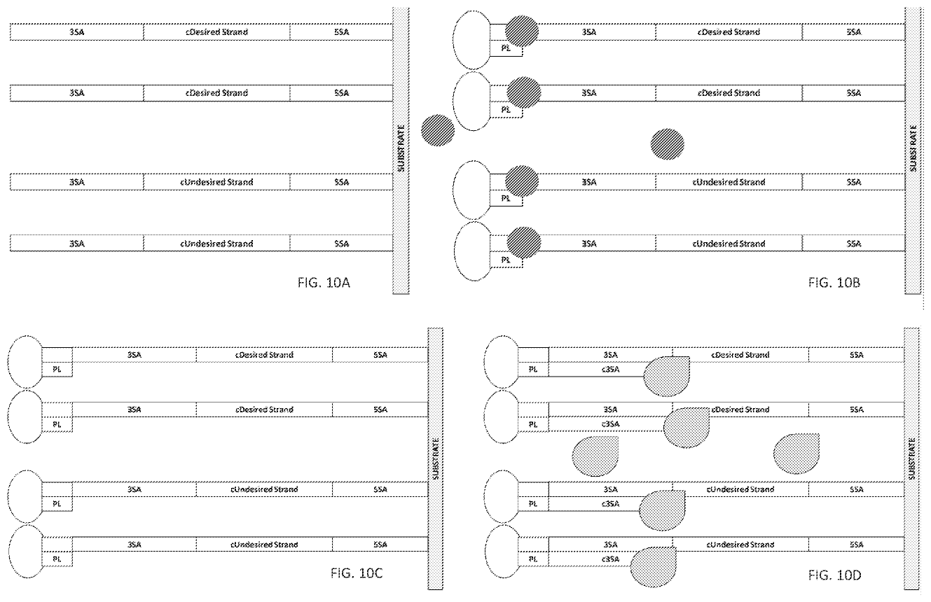

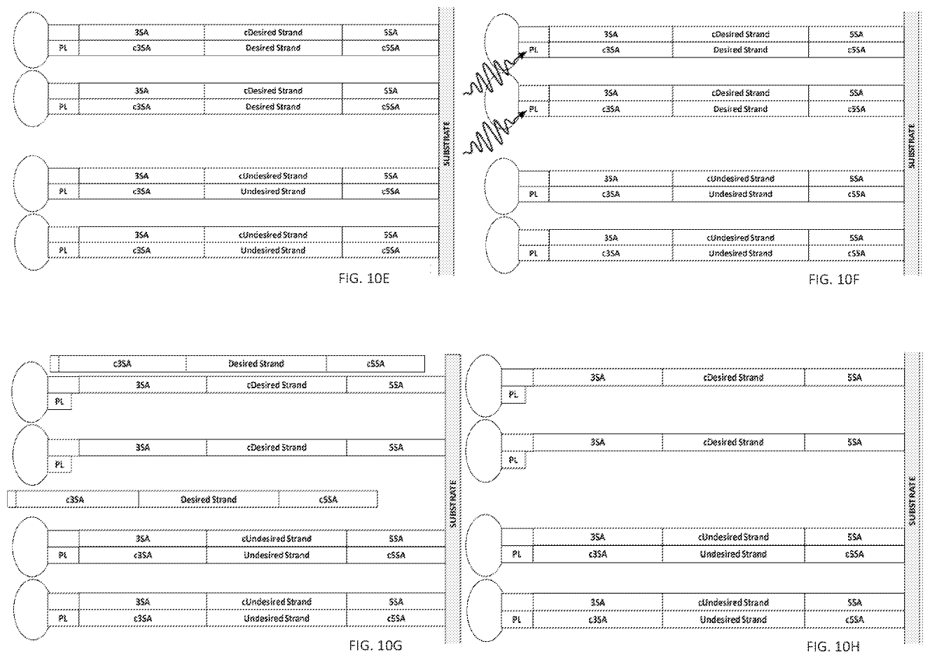

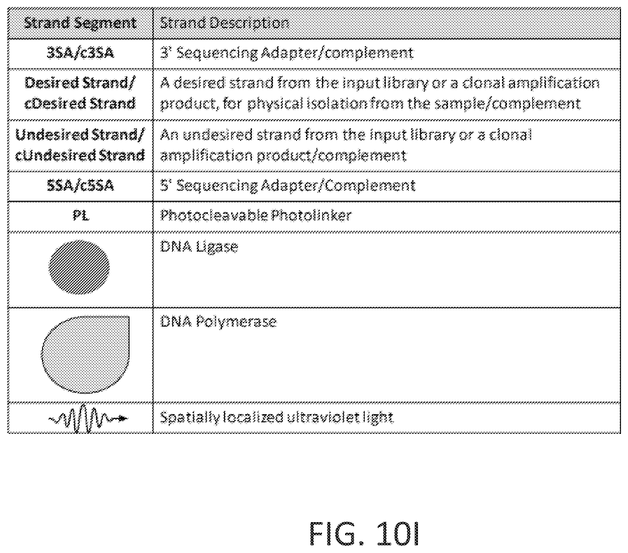

A substrate-based method of isolating desired nucleic acid strands from a mixed library is described in this example. A sample comprising the mixed library can be provided to a substrate. The substrate can comprise a plurality of nucleic acids bound to distinct locations on the surface of the substrate, without the use of cleavable linkers. The sequence of the nucleic acid strands bound to the substrate can be identified, and each strand can be identified as accurate or inaccurate or as desired or undesired. After sequencing strands attached to a flow cell with substrate-based sequencing (SBS), the extended sequencing primer can be melted from the substrate-bound template strand and washed out of the flow cell (FIG. 10A). A 5-phosphorylated hairpin oligonucleotide containing a photocleavable linker between two nucleotides on its 3′-end can be attached to the 3′-ends of all template strands with DNA ligase (FIG. 10B). Next, the 3′-end of the primer can be extended with DNA polymerase (FIG. 10D), resulting in replication of the sequenced template strand that is attached to the flow cell (FIG. 10E). Finally, the desired strands can be selectively exposed to UV light (FIG. 10F), resulting in cleavage of the photocleavable linker in the extended hairpin oligonucleotide (FIG. 10G). At this point, a strand that is the desired strands can be selectively isolated by chemical or thermal melting and extracting the resulting solution from the flow cell (FIG. 10G), resulting in the retention of the undesired strands on the substrate (FIG. 10H). A legend for the above figures is shown in FIG. 10I.

### Example 7

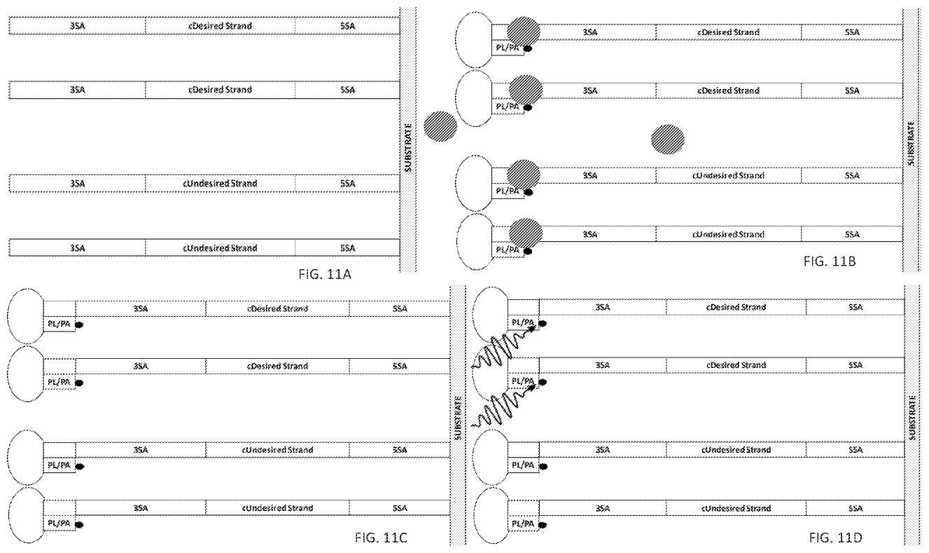

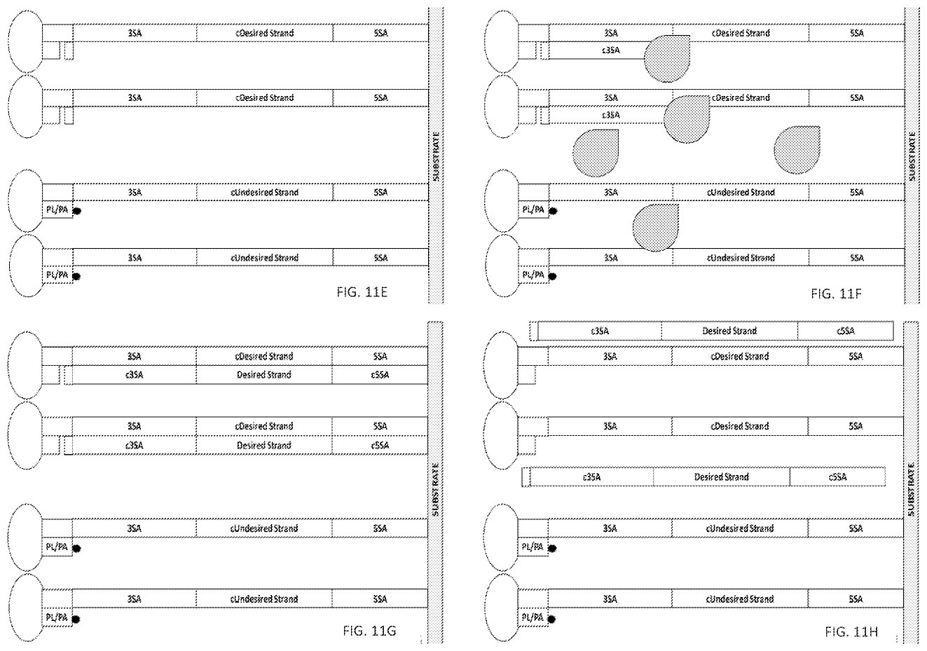

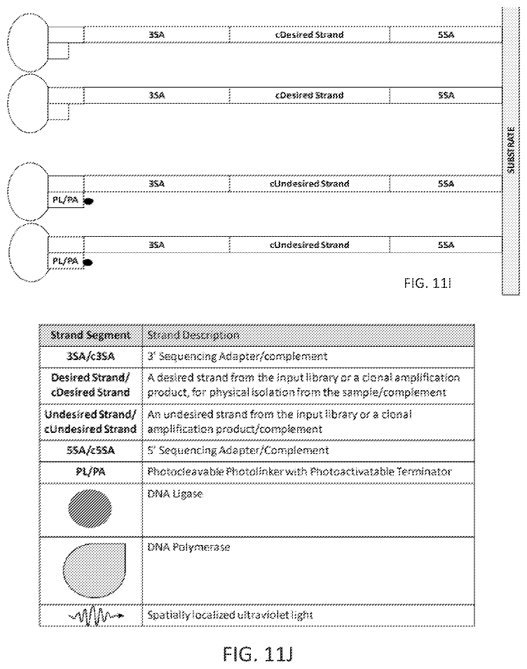

A substrate-based method of isolating desired nucleic acid strands from a mixed library is described in this example. A sample comprising the mixed library can be provided to a substrate. The substrate can comprise a plurality of nucleic acids bound to distinct locations on the surface of the substrate, without the use of cleavable linkers. The sequence of the nucleic acid strands bound to the substrate can be identified, and each strand can be identified as accurate or inaccurate. After sequencing strands attached to a flow cell with substrate-based sequencing (SBS), the extended sequencing primer can be melted from the substrate-bound template strand and washed out of the flow cell (FIG. 11A). A 5-phosphorylated hairpin oligonucleotide containing a photocleavable linker between two nucleotides near its 3′-end and a photoactivatable or photo-reversible terminator at its 3′-end can be attached to the 3′-ends of all template strands with DNA ligase (FIG. 11B). Next, the desired strands can be selectively exposed to UV light (FIG. 11D), resulting in cleavage of the photocleavable linker and reversion of the photoactivatable or photoreversible terminator to a form that allows primer extension by DNA polymerase. Next, the 3′-end of the primer can be extended with DNA polymerase (FIG. 11F), resulting in replication of the sequenced template strand that is attached to the flow cell only for the desired strand (FIG. 11G). At this point, the desired strands can be selectively isolated by chemical or thermal melting and extracting the resulting solution from the flow cell (FIG. 11H), resulting in the retention of the undesired strands on the substrate (FIG. 11I). A legend for the above figures is shown in FIG. 11J.

### Example 8

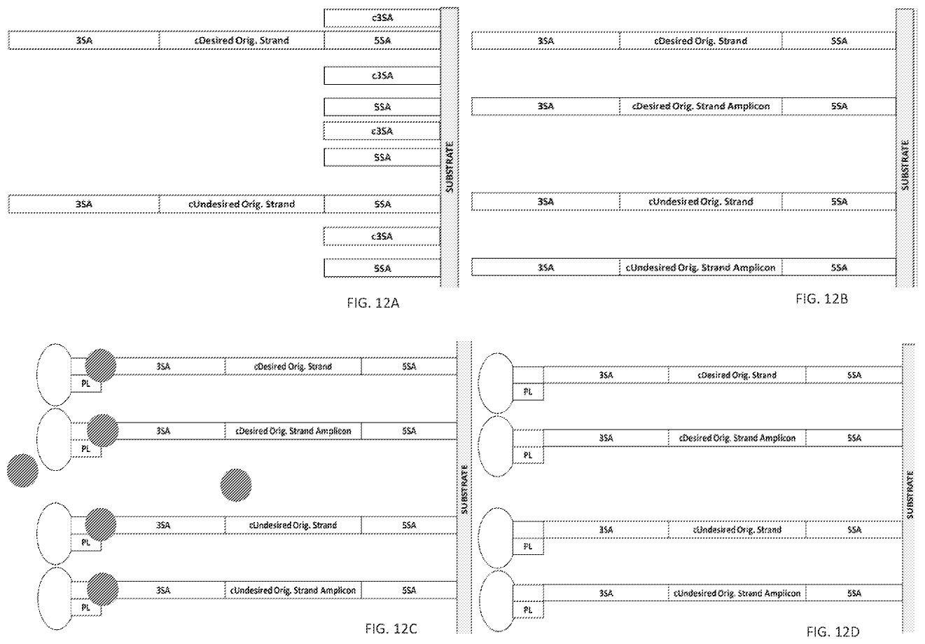

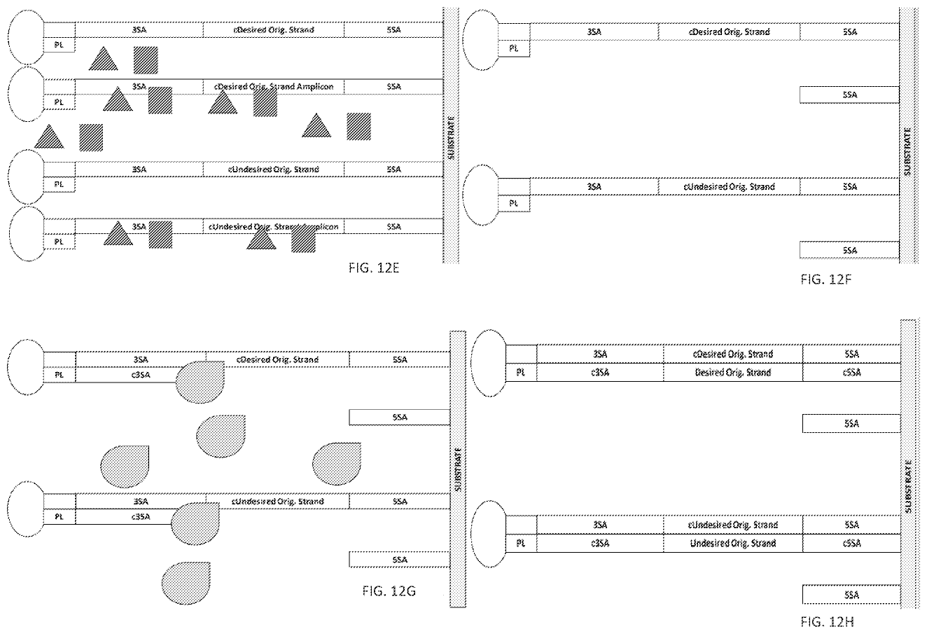

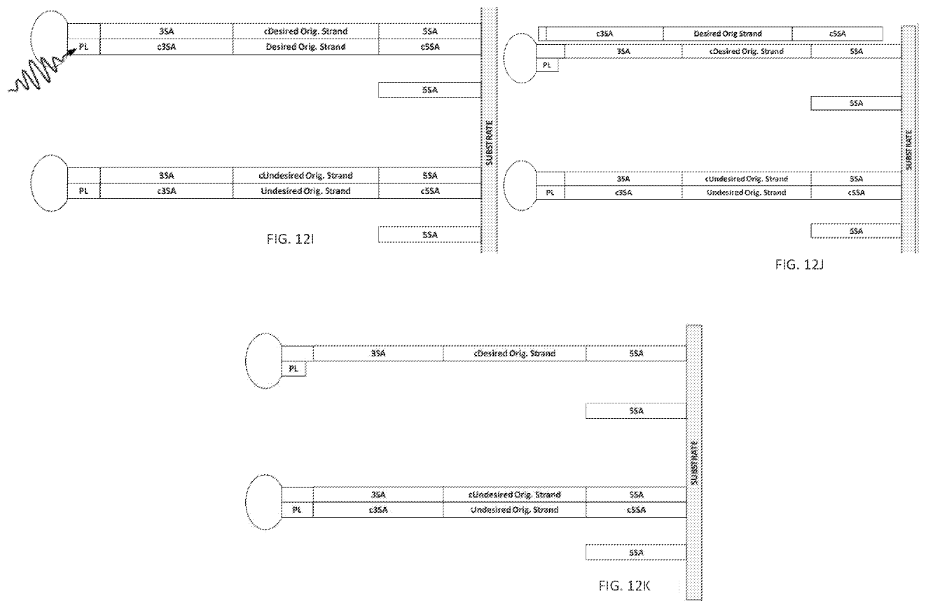

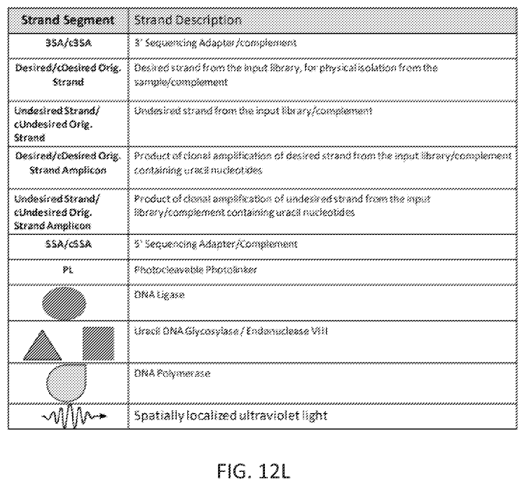

A substrate-based method of isolating desired nucleic acid strands from a mixed library is described in this example. While clonal amplification of the library strands is used for sequencing, in this example, only the original, desired strands are isolated, rather than a mixture of amplicons of the original, desired strands and/or the original, desired strands. A sample comprising the mixed library can be provided to a substrate. The substrate can comprise a plurality of nucleic acids bound to distinct locations on the surface of the substrate, without the use of cleavable linkers. The nucleic acid strands can be replicated by DNA polymerase, resulting in covalent attachment of their complements to the substrate (FIG. 12A). The nucleic acid strands can then be clonally amplified by solid-phase amplification (e.g. bridge PCR or related methods), substituting dUTP for dTTP in the mixture of nucleotides used by DNA polymerase. This results in clonal amplicons containing dU, dA, dG, and dC nucleotide bases, whereas the original strand contains dT, dA, dG, and dC nucleotide bases (FIG. 12B). The sequence of the nucleic acid strands bound to the substrate can be identified, and each strand can be identified as accurate or inaccurate. After sequencing strands attached to a flow cell with substrate-based sequencing (SBS), the extended sequencing primer can be melted from the substrate-bound template strand and washed out of the flow cell. A 5′-phosphorylated hairpin oligonucleotide containing a photocleavable linker between two nucleotides on its 3′-end can be attached to the 3′-ends of all template strands with DNA ligase (FIG. 12C). Next, a mixture of uracil DNA deglycosylase and endonuclease VIII (i.e. USER enzyme mixture) can be introduced, destroying all of the clonal amplification products on the flow cell. The enzyme mixture deglycosylates dU nucleotides, which are absent from the original template strands, and digests strands containing deglycosylated nucleotides (FIG. 12E). Next, the 3′-end of the primer is extended with DNA polymerase (FIG. 12G), resulting in replication of the sequenced template strand that is attached to the flow cell (FIG. 12H). Finally, the desired strands can be selectively exposed to UV light (FIG. 12I), resulting in cleavage of the photocleavable linker in the extended hairpin oligonucleotide (FIG. 12J). At this point, the original desired strand can be selectively isolated by chemical or thermal melting and extracting the resulting solution from the flow cell (FIG. 12J), resulting in the retention of the undesired strands on the substrate (FIG. 12K). A legend for the above figures is shown in FIG. 12L.

### Example 9

A substrate-based method of isolating accurate nucleic acid strands from a mixed library is described in this example. A sample comprising the mixed library can be provided to a substrate. The substrate can comprise a plurality of nucleic acids bound to distinct locations on the surface of the substrate, without the use of cleavable linkers. The sequence of the nucleic acid strands bound to the substrate can be identified, and each strand can be identified as accurate or inaccurate. After sequencing strands attached to a flow cell, such as with substrate-based sequencing (SBS), the extended sequencing primer is melted from the substrate-bound template strand and washed out of the flow cell. Universal primers are then introduced and extended with DNA polymerase, resulting in a complementary strand for each sequenced strand. Desired strands are then selectively melted from the surface by applying spatially localized heating, and are extracted from the flow cell.

It is understood that the foregoing detailed description and accompanying examples are merely illustrative and are not to be taken as limitations upon the scope of the disclosure, which is defined solely by the appended claims and their equivalents. All publications and patents mentioned in the above specification are herein incorporated by reference as if expressly set forth herein. Various changes and modifications to the disclosed embodiments will be apparent to those skilled in the art and may be made without departing from the spirit and scope thereof.

What is claimed is:

1. A method of separating desired nucleic acid molecules from undesired nucleic acid molecules contained in a mixed library of nucleic acid molecules, comprising: a) sequencing individual nucleic acid molecules within said mixed library at a localized zone of a device; and b) selectively separating desired nucleic acid from undesired nucleic acid by releasing either the desired or the undesired nucleic acid from said localized zone based on its determined sequence.

2. The method of claim 1, wherein the nucleic acid molecules are synthesized nucleic acid molecules.

3. The method of claim 1, comprising separating a first population of desired nucleic acid molecules into a first sub-library, and separating a second population of desired nucleic acid molecules into a second sub-library.

4. A method of isolating desired nucleic acid strands from a mixed library, the method comprising:
a) providing a sample containing the mixed library to a first chamber of a nanopore sequencing device, the device comprising a first chamber and a second chamber separated by a substantially impermeable membrane housing a plurality of nanopores:
b) inducing a flow of current through each nanopore, such that individual nucleic acid strands enter into the nanopores housed within the membrane:
c) determining the sequence of each individual nucleic acid strand as it passes through a nanopore and identifying each strand as accurate or inaccurate; and
d) isolating the desired nucleic acid strands from the sample.

5. The method of claim 4, wherein the nanopore sequencing device comprises a plurality of electrodes, wherein each electrode is operably connected to a distinct nanopore within the substantially impermeable membrane.

6. The method of claim 5, wherein inducing a flow of current through each nanopore comprises applying a voltage through each of the plurality of electrodes.

7. The method of claim 5 or claim 6, wherein the nanopore sequencing device further comprises a plurality of sensors, wherein each sensor records a current passing through a single nanopore, such that the current passing through each nanopore is independently recorded.

8. The method of claim 7, wherein determining the sequence of each individual nucleic acid strand as it passes through a nanopore comprises:
a) recording the current passing through each nanopore; and
b) determining the sequence of a given nucleic acid strand based upon the disruption of current that occurs as the nucleic acid strand passes through the nanopore.

9. The method of claim 8, wherein identifying each strand as accurate or inaccurate comprises comparing the determined sequence of a given nucleic acid strand to an accurate nucleic acid sequence.

10. The method of claim 9, wherein a strand comprising one or more mutations compared to the accurate nucleic acid sequence is identified as an inaccurate strand.

11. The method of claim 10, wherein isolating the desired nucleic acid strands from the sample comprises modulating the voltage applied through each electrode operably connected to a nanopore containing an undesired nucleic acid strand, such that passage of the undesired nucleic acid strand through the nanopore is halted and/or reversed.

12. The method of claim 11, wherein the voltage applied through each electrode operably connected to nanopore containing a desired nucleic acid strand is not modulated, such that desired nucleic acid strands pass through the nanopores into the second chamber of the nanopore sequencing device.

13. The method of claim 12, further comprising isolating the desired nucleic acid strands from the second chamber of the nanopore sequencing device.

14. The method of claim 12, further comprising reversing the voltage applied to each electrode operably connected to a nanopore containing an undesired nucleic acid strand, such that the undesired nucleic acid strands are ejected into the first chamber of the nanopore sequencing device.

15. The method of claim 14, further comprising removing the undesired nucleic acid strands from the first chamber, followed by reversing the voltage applied to one or more electrodes, such that the desired nucleic acid strands housed within the second chamber are drawn through the nanopores into the first chamber.

16. The method of claim 15, further comprising removing the desired nucleic acid strands from the first chamber.

17. The method of any one of the preceding claims, wherein one or more steps of the method are performed using a computer.

18. A method of isolating accurate nucleic acid strands from a mixed library, the method comprising:
a) providing a sample comprising the mixed library to a substrate, such that individual nucleic acid strands bind to the substrate:
b) sequencing the nucleic acid strands:
c) applying a stimulus to selectively release desired nucleic acid strands from the substrate; and
d) isolating the released nucleic acid strands.

19. The method of claim 18, wherein the stimulus comprises heat.

20. The method of claim 18, further comprising adding photocleavable linkers to desired nucleic acid strands after step b) and prior to step c)

21. The method of claim 20, wherein the stimulus comprises light, wherein the light selectively cleaves the photocleavable linkers added to the desired nucleic acid strands, thereby releasing the desired nucleic acid strands from the substrate.

22. The method of claim 21, wherein the stimulus comprises multi-photon excitation.

23. The method of claim 22, wherein the multi-photon excitation is two-photon excitation or three-photon excitation.

24. A method of isolating desired nucleic acid strands from a mixed library, the method comprising:
a) providing a sample comprising the mixed library to a substrate, the substrate comprising a plurality of cleavable anchors at distinct locations on the surface of the substrate, such that individual nucleic acid strands bind to the cleavable linkers:
b) applying a stimulus to induce selective cleavage of the cleavable anchors bound to desired locations on the surface of the substrate, thereby releasing nucleic acid strands from the surface of the substrate; and
c) isolating the released nucleic acid strands.

25. The method of claim 24, further comprising determining the sequence of the nucleic acid strands bound to the cleavable linkers, and identifying each strand as accurate or inaccurate.

26. The method of claim 25, wherein applying a stimulus to induce selective cleavage of the cleavable anchors comprises applying a stimulus to induce selective cleavage of the cleavable anchors bound to desired nucleic acid strands, thereby releasing the desired nucleic acid strands from the surface of the substrate.

27. The method of claim 25 or claim 26, wherein identifying each strand as accurate or inaccurate comprises comparing the determined sequence of a given nucleic acid strand to an accurate nucleic acid sequence.

28. The method of claim 27, wherein a strand comprising one or more mutations compared to the accurate nucleic acid sequence is identified as an inaccurate strand.

29. The method of any one of claims 24-28, wherein the cleavable anchors are photocleavable and wherein the stimulus to induce selective cleavage is light.

30. The method of claim 29, wherein the light is applied to specific spatial locations on the substrate.

31. The method of claim 29 or claim 30, wherein the stimulus comprises multi-photon excitation.

32. The method of claim 31, wherein the multi-photon excitation is two-photon excitation or three-photon excitation.

33. The method of any one of claims 24-28, wherein the cleavable anchors are heat-sensitive and wherein the stimulus to induce selective cleavage is heat.

34. The method of claim 33, wherein the heat is applied to specific spatial locations on the substrate.

35. A system for isolating desired nucleic acid strands from a mixed library, the system comprising a sequencing device and software, wherein the software collects data from the sequencing device, analyzes the data, and actuates components of the system to control the isolation of accurate nucleic acids from the mixed library.

36. The system of claim 35, wherein collecting data comprises determining the sequence of a nucleic acid at a localized zone of the sequencing device and wherein analyzing the data comprises comparing the sequence of the nucleic acid to the sequence of a desired nucleic acid strand.

37. The system of claim 35 or 36, wherein the software encodes machine readable instructions that instruct a processor to execute a given task to control the isolation of accurate nucleic acids from the mixed library.

38. The system of claim 37, wherein the software encodes machine readable instructions that instruct a processor to apply a stimulus that results in selective release of either a desired or an undesired nucleic acid strand from the localized zone of the sequencing device.

39. The system of any one of claims 35-38, wherein the sequencing device is a nanopore based sequencing device.

40. The system of claim 39, wherein the software encodes machine readable instructions that instruct a processor to apply a voltage or to modulate voltage at a given electrode of the nanopore based sequencing device, thereby selectively releasing either a desired or an undesired nucleic acid strand from a nanopore operably connected to the electrode.

41. The system of any one of claims 35-38, wherein the sequencing device is a substrate-based sequencing device.

42. The system of claim 35, wherein the software encodes machine readable instructions that instruct a processor to apply a stimulus to a defined spatial location on a substrate, thereby releasing either a desired or an undesired nucleic acid strand from the defined spatial location on the substrate.
